(19) United States
(12) Patent Application Publication
(10) Pub. No.: US 2006/0252051 A1
(43) Pub. Date: Nov. 9, 2006

(54) Method for producing diverse libraries of encoded polymers

(76) Inventors: Charles E. Merryman, Sykesville, MD (US); D. Rachel Green, Baltimore, MD (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/235,667

(22) Filed: Sep. 26, 2005

Related U.S. Application Data
(63) Continuation of application No. PCT/US04/09648, filed on Mar. 29, 2004.
(60) Provisional application No. 60/458,192, filed on Mar. 27, 2003.
(60) Provisional application No. 60/535,781, filed on Jan. 12, 2004.

(51) Int. Cl. C12Q 1/68; C07H 21/02; C07H 21/04

(52) U.S. Cl. 435/6; 536/23.1; 536/25.3

(57) ABSTRACT

Described are aminoacyl tRNA analogues which comprise a tRNA, and an amino acid which acts as an acceptor and donor substrate for ribosome-directed translation, thus, incorporating unusual monomers into non-standard polymers by the action of ribosomes. Also described are methods for producing such tRNA analogues; non-standard polymers; libraries of encoded polymers; methods of screening the libraries; and target members and their uses. A key advantage of synthesizing non-standard polymer libraries of the present invention with aminoacyl tRNA analogues is that large libraries of high complexity can be easily made and functional library members (e.g. novel drugs) readily identified.

### RELATED APPLICATIONS

This application is a continuation of International Application No. PCT/US2004/009648, which designated the United States and was filed on Mar. 29, 2004, published in English, which claims the benefit of U.S. Provisional Application No. 60/458,192, filed on Mar. 27, 2003 and U.S. Provisional Application No. 60/535,781, filed on Jan. 12, 2004. The entire teachings of the above applications are incorporated herein by reference.

### GOVERNMENT SUPPORT

The invention was supported, in whole or in part, by a grant RO1 GM 59425 from the National Institutes of Health. The Government has certain rights in the invention.

### BACKGROUND OF THE INVENTION

Large combinatorial libraries of polymers are starting points for isolating new catalysts, binding motifs and other useful molecules. For example, current evolutionary approaches can generate populations of nucleic acids with complexities on the order of 10¹⁵ molecules from which a single molecule with a desired activity is then isolated and identified. Proteins have greater chemical diversity than do polynucleotides, making them an attractive alternative to nucleic acids, and several methods allow functional sequences in protein libraries to be identified by evolutionary approaches (Roberts, et al., US published patent application 2003/0235851A1; Gold, U.S. Pat. No. 5,843,701; Merryman, et al., Chem. & Biol. 9:741-746 (2002); Merryman and Bartel, U.S. Pat. No. 6,440,695) However, proteins are susceptible to biodegradation and are limited to a small set of monomers. Thus, it would be useful to be able to produce protein-like or polypeptide-like libraries of non-standard polymers that have alterations in the polymer backbone, potentially rendering the non-standard polymers resistant to biodegradation, for example, by proteases.

Unusual amino acids can be used by aminoacyl tRNA synthetases potentially broadening the range of available monomers. However, such alterations rarely alter the structure of the polypeptide backbone. Thus, polypeptides containing such unusual amino acids are virtually equivalent to peptide or protein libraries. In addition, to achieve high diversity libraries that are suitable for drug discovery (M.W. ˜1000; chain length ˜10), about 20 different building blocks (such as aminoacylated tRNAs) are required (10¹³˜20¹⁰). Because mRNA encoding systems produce about 10¹¹ to 10¹² library members per milliliter, a diverse library of 10¹³ would require a translation reaction as large as 100 ml. At normal in vitro protein synthesis concentrations (1 mg/ml tRNA), a considerable amount of tRNA would be necessary (10-100 mg). Unfortunately, current chemical misacylation techniques depend on individually isolated tRNAs, separate reactions, multiple steps, and commercially unavailable reagents (Noren, C. J., et al., Science 244:182-188 (1989); Bain, J. D., et al., Tetrahedron 47: 2389-2400 (1991); Mendel, D., et al., J. Am. Chem. Soc. 115:4359-4360 (1993); Thorson, J. S., et al., Meth. Mol. Biol. 77:43-73 (1998); Hohsaka, T., et al., J. Am. Chem. Soc. 121, 34-40 (1999)). Furthermore, a tRNA is consumed for every codon translated (a necessity when enzymes are not capable of recharging expended tRNAs within a translation reaction), limiting library complexity by the amount of non-standard aminoacyl-tRNA that can be made.

It would be useful to be able to apply protein evolutionary approaches to non-standard or non-natural polymers. However, current systems for producing aminoacylated tRNAs are limited in their ability to generate both sufficient quantities of misacylated tRNAs as well as large complex libraries of misacylated tRNAs. This fundamental problem limits the ability to generate derivative libraries of non-standard polymers.

### SUMMARY OF THE INVENTION

The present invention is drawn to methods for producing an aminoacyl tRNA analogue comprising a tRNA and a modified amino acid which acts as an acceptor substrate and a donor substrate for ribosome-directed translation. In one embodiment, an aminoacylated tRNA analogue represented by the following structural formula: 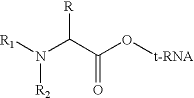
is produced; wherein R is an amino acid side chain; R₁ is H or an amine protecting group; and R₂ is a group other than H, such that the compound represented by the following formula acts as an acceptor substrate and a donor substrate for ribosome-directed translation: 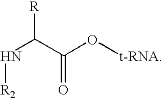
The method comprises the step of reacting a starting compound with a reagent capable of converting R₁—NH—R″ to R₁—NR₂—R″, wherein R″ is an organic moiety, under conditions suitable for said reaction, wherein the starting compound is represented by the following structural formula:

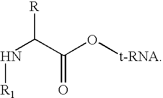

In another embodiment, the method involves preparing an aminoacylated tRNA analogue represented by the following structural formula: 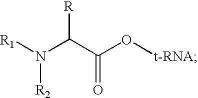
wherein R is an amino acid side chain; R₁ is H or an amine protecting group; and R₂ is an alkyl such that the compound represented by the following formula acts as an acceptor substrate and a donor substrate for ribosome-directed translation. 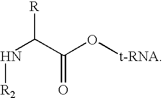
The method comprises the step of alkylating a starting compound represented by the following structural formula:

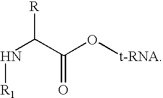

In another embodiment, the method comprises substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue.

In another embodiment, a tRNA, an amino acid, and an aminoacyl tRNA synthetase are combined under conditions in which an aminoacylated tRNA is formed. The aminoacyl tRNA is substituted under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue.

The present invention is also drawn to methods for producing a population of tRNAs comprising aminoacyl tRNAs and aminoacyl tRNA analogues, wherein the aminoacyl tRNA analogues comprise a tRNA and a modified amino acid such that the aminoacyl tRNA analogues act as acceptor substrates and donor substrates for ribosome-directed translation.

In one embodiment, the method comprises combining a plurality of tRNAs that are specific for a plurality of amino acids, amino acids which are specific for a subset of the tRNAs, and aminoacyl tRNA synthetases, under conditions in which the subset of tRNAs are aminoacylated, thereby forming a mixture of tRNAs that are aminoacylated and tRNAs that are not aminoacylated. The tRNAs that are aminoacylated are substituted under conditions in which the α-amino group of each aminoacylated tRNA is monosubstituted with a group other than H, thereby forming aminoacyl tRNA analogues which act as acceptor substrates and donor substrates for ribosome-directed translation. The mixture is then contacted with amino acids that are specific for the tRNAs that are not aminoacylated, under conditions in which aminoacylated tRNAs are formed, thereby producing the population of tRNAs comprising aminoacyl tRNAs and aminoacyl tRNA analogues.

The present invention is also drawn to methods for producing a non-standard polymer. The method comprises producing an aminoacyl tRNA analogue (one or more) comprising substituting an aminoacyl tRNA under conditions in which the α-amino group of the aminoacylated tRNA is monosubstituted with a group other than H, such that the aminoacyl tRNA analogue acts as an acceptor substrate and a donor substrate for ribosome-directed translation. The aminoacyl tRNA analogue (one ore more) is combined with encoding nucleic acid and an in vitro translation mixture and maintained under conditions in which the aminoacyl tRNA analogue is used in the translation of the encoding nucleic acid, thereby producing a non-standard polymer. The aminoacyl tRNA analogue can also be produced separately, according to the method of the present invention for producing aminoacyl tRNA analogues, and combined with encoding nucleic acid and an in vitro translation mixture. In this method, one or more tRNA analogues can be used to produce the non-standard polymer. For example, the method can comprise using a tRNA analogue that comprises an N-methyl amino acid and a tRNA that comprises an N-ethyl amino acid, wherein the N-methyl and N-ethyl amino acids are incorporated into the non-standard polymer.

The present invention is also drawn to methods for producing a library comprising non-standard-polymers. The method comprises producing an aminoacyl tRNA analogue (one or more) comprising substituting an aminoacyl tRNA under conditions in which the α-amino group of the aminoacylated tRNA is monosubstituted with a group other than H, such that the aminoacyl tRNA analogue acts as an acceptor substrate and a donor substrate for ribosome-directed translation. The aminoacyl tRNA analogue (one or more) is combined with a population of encoding nucleic acids and an in vitro translation mixture and maintained under conditions in which the aminoacyl tRNA analogue is used in the translation of the encoding nucleic acids, thereby producing a library comprising non-standard polymers. The aminoacyl tRNA analogue can also be produced separately, according to the method of the present invention for producing aminoacyl tRNA analogues, and combined with encoding nucleic acid and an in vitro translation mixture.

The present invention is also drawn to methods for identifying a non-standard polymer having a desired activity. The method comprises producing a library comprising non-standard polymers. To produce the library comprising non-standard polymers, an aminoacyl tRNA analogue (one or more) is produced, comprising substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation. The aminoacyl tRNA analogue (one or more) is combined with a population of encoding nucleic acids and an in vitro translation mixture and maintained under conditions in which the aminoacyl tRNA analogue is used in the translation of the encoding nucleic acids, thereby producing a library comprising non-standard polymers. A member of the library that exhibits a desired activity is selected, thereby identifying a non-standard polymer having a desired activity. The aminoacyl tRNA analogue can also be produced separately, according to the method of the present invention for producing aminoacyl tRNA analogues, and combined with encoding nucleic acids and an in vitro translation mixture.

The present invention is also drawn to tRNA analogues produced by the method of the present invention. In one embodiment, the aminoacyl tRNA analogue comprises a tRNA and a modified amino acid, wherein an et-amino group of the amino acid is monosubstituted with a lower alkyl group, and wherein if the α-amino group is monosubstituted with a methyl group, the amino acid is other than leucine, glycine, phenylalanine or alanine, wherein the aminoacyl tRNA analogue acts as an acceptor substrate and a donor substrate for ribosome-directed translation.

In another embodiment, the present invention is drawn to a population of aminoacyl tRNA analogues wherein each aminoacyl tRNA analogue comprises a tRNA and a modified amino acid wherein an α-amino group of each amino acid is monosubstituted with a lower alkyl group, and wherein the amino acids include alanine, arginine, asparagine, aspartic acid, cysteine, glutamine, glutamic acid, glycine, histidine, isoleucine, lysine, methionine, phenylalanine, serine, threonine, tryptophan, tyrosine, and valine, wherein the aminoacyl tRNA analogues act as acceptor substrates and donor substrates for ribosome-directed translation.

The present invention is also drawn to an aminoacyl tRNA analogue comprising a tRNA and a modified amino acid, wherein an α-amino group of the amino acid is monosubstituted with a protecting group selected from the groups consisting of: o-nitrophenyl and substituted versions thereof.

In another embodiment, the aminoacyl tRNA analogue comprises a tRNA and a modified amino acid, wherein an α-amino group of the amino acid is substituted with a protecting group and a lower alkyl group.

The present invention allows ribosomal synthesis of non-standard polymers on a level sufficient to generate searchable libraries. Ribosome synthesis allows for the encoding of products with nucleic acid and the subsequent processing of staggeringly complex libraries (>10¹³) by in vitro selection of the non-standard polymer library and amplification of their encoding nucleic acids. The unique power of evolutionary methods is the ease with which library members having a desired function can be identified. The basic procedure is to use iterative rounds of selection and amplification, where the selective step increases the proportion of molecules having a desired characteristic, and amplification increases their number. With each round, the library is exponentially enriched in molecules that satisfy the selective criteria. Thus, an originally diverse population that may contain only a single copy of a desirable molecule quickly evolves into a population dominated by the molecule.

As described herein, readily available and inexpensive bulk tRNA can be used to generate aminoacyl tRNA analogues comprising a tRNA and a modified amino acid that acts as an acceptor substrate and a donor substrate for ribosome-directed translation. tRNA synthetases ensure that the correct amino acids are attached to their cognate tRNAs prior to modification. Subsequent chemical transformation of the whole population of aminoacyl tRNA(s) then allows for the immediate synthesis of aminoacyl tRNA comprising altered monomers that can be used in translation. The bulk tRNA can be fractionated into subpopulations of tRNA prior to modification according to the method of the present invention, thus allowing the generation of any combination of desired aminoacyl tRNA analogues. The chemical manipulations described herein proceed in slightly acidic, aqueous media under conditions which do not inactivate the 25 kDa tRNAs to which the amino acids are attached.

The transformation described herein is quantitative. Furthermore, because this approach can be used with bulk aminoacylated tRNA, it is trivial to simultaneously synthesize large amounts of numerous N-alkyl aminoacyl tRNAs, such as N-methyl tRNAs. By eliminating the substantial synthetic efforts demanded by traditional methods, the present invention provides a readily accessible means for producing and searching vast libraries (˜10¹³) of “drug-like” non-standard polymers via ribosome-directed translation and in vitro selection.

Beyond the use of N-methylated or N-alkylated aminoacyl tRNAs for in vitro selections, the methods described herein and aminoacyl tRNA analogues produced by the methods can be used in other endeavors involving peptides and proteins. For example, the aminoacyl tRNA analogues of the present invention can be used in conjunction with nonsense suppression (Noren C J, Anthony-Cahill S J, Griffith M C, Schultz P G., Science 244:182-188 (1989). Again, a major benefit of the present invention over current technology is the ease with which N-methylated aminoacyl tRNAs can now be made. For example, biophysical studies of proteins often require quantities of material that exceed all but the most dedicated attempts, as current chemical aminoacylation methods involve “ . . . a borderline heroic effort, . . . ” (Dougherty D A, Curr Opin Chem Biol. 4:645-652 (2000). At the same time, the overall contribution of main chain hydrogen bonds to protein stability and activity are not well understood. Our methods allow the production of vast amounts of N-methylated aminoacyl tRNA; and therefore, allow comparatively easy production of proteins where individual or multiple main-chain hydrogen bonds are interrupted. The present invention allows examination of fundamental aspects of protein structure, function, and folding. It would also now be possible to scan residues of existing functional peptides or proteins with N-methylated residues. In an appropriate screen or selection, backbone nitrogens that could be methylated without changing the function of the parent molecule could be identified. Such derivatives are likely to have improved stability or permeability resulting in enhanced function. Another application would be in controlling the release of existing peptides or proteins. Protease or peptidase activity is often connected to the production of the active form of a peptide or protein, and the appropriate placement of methylated residues has the potential to slow the rate of cleavage without completely preventing it. This sort or experimentation would likely result in more effective constant availability of a protein or peptide rather than a bolus of activity. The methods described herein allow the substrates required for such experiments to be made with essentially no effort or expense and thus greatly increase the extent and scope their application.

### BRIEF DESCRIPTION OF THE DRAWINGS

FIG. 1A is a schematic of an aminoacylated tRNA molecule including SEQ ID NO: 1, where D is dihydrouridine (d), Y is wybutosine (wybutine, yw), Ψ is pseudouridine (p) and T is 5-methyluridine (t).

FIG. 1B is a schematic representation of translation of a mRNA molecule.

FIG. 2 is a schematic representation of one embodiment of the present invention, where in step 1 tRNA is aminoacylated by an aminoacyl tRNA synthetase, in step 2, the aminoacyl tRNA is protected with a nitrophenyl group, in step 3, the protected aminoacyl tRNA is substituted by alkylation, and in step 4, the amine is deprotected, resulting in a N-methyl-tRNA.

FIG. 3 is a schematic representation of in vitro translation where every codon in an encoding nucleic acid is decoded by a N-methyl-tRNA, thus forming a non-standard polymer that is wholly polymerized from N-methyl-tRNAs.

FIG. 4 shows an outline of the steps used for N-methylation of enzymatically charged tRNA.

FIG. 5A shows the structure of puromycin where the reactive primary amine of the amino acid moiety (methoxytyrosine) is boxed.

FIG. 5B is a gel showing the synthesis of N-monomethyl puromycin, where the products of the reaction were separated by TLC, visualized by UV shadowing and photographed: lane 1, puromycin; lane 2, N-nitrophenyl puromycin; lane 3, N-nitrophenyl-N-methyl puromycin; lane 4, N-methyl puromycin; lane 5, N,N-dimethyl puromycin.

FIG. 6A is a gel showing the synthesis of N-monomethyl-Glu, Ser and Val, where the products and intermediates of the reaction were separated by TLC and visualized by phosphorimager analysis. In lanes 2-8, the aminoacyl ester bond was hydrolyzed to free the modified amino acid from its immobile tRNA, and lane 8 employed bulk tRNA that was charged with a complete amino acid mix and a single radiolabeled amino acid.

FIG. 6B is a gel showing comigration of N-methyl amino acid standards and the indicated transformed amino acids.

FIG. 7A is a gel showing in vitro translation products of an 88 codon transcript of β-galactosidase using bulk aminoacyl tRNA (lanes 1, 2, 4, and 6), where lane 1 did not contain the transcript, and using bulk tRNA that had been mock treated prior to aminoacylation (lanes 3, 5, and 7).

FIG. 7B is a gel showing dipeptide formation with mock-treated tRNA. From left to right, the lanes for each amino acid used normal tRNA or mock-treated tRNA.

FIG. 8 shows gels which demonstrate peptide acceptor activity of tRNA bearing methylated Ser or Val, wherein lanes 2 and 4 ribosomes were supplied within RNA and fMET-tRNA^{met} and N-methyl-aminoacyl tRNA to form dipeptides, and lanes 1 and 3 are controls lacking mRNA. In lanes 1 and 2, the initiator tRNA was labeled with [³⁵S]-Met.

FIG. 9 shows gels which demonstrate acceptor activity of bulk aminoacyl tRNA. Translation initiation complexes containing f-[³⁵S]-Met-tRNA^{met} and an appropriate mRNA (see Experimental Procedures) were supplied bulk aminoacyl tRNA, and the dipeptides produced were separated by eTLC. From left to right, the lanes for each amino acid used normal tRNA, N-methyl tRNA, or N,N-dimethyl tRNA as the acceptor substrate. The dipeptide (Di) and basic dipeptide (bDi) products are indicated at right.

FIG. 10 is a gel showing random pentapeptides produced using normal aminoacyl tRNAs (lanes 1 and 2), and using N-methyl aminoacyl tRNA analogues produced by the method of the present invention (lanes 3 and 4), where lanes 2 and 4 were treated with proteinase K.

### DETAILED DESCRIPTION OF THE INVENTION

The present invention is drawn to methods for producing aminoacyl tRNA analogues comprising a tRNA and a modified amino acid which acts as an acceptor substrate and a donor substrate for ribosome-directed translation, and methods of producing non-standard polymers and libraries of non-standard polymers using the aminoacyl tRNA analogues. The invention is also drawn to methods of screening a library comprising non-standard polymers for members having a desired activity, wherein the non-standard polymers are produced using the aminoacyl tRNA analogues. The invention is also drawn to tRNA analogues comprising a tRNA and a modified amino acid which acts as an acceptor substrate and a donor substrate for ribosome-directed translation. A key advantage of synthesizing non-standard polymer libraries with the aminoacyl tRNA analogues of the present invention is that very large libraries can be easily made because mixtures of the aminoacyl tRNA analogues of the present invention are easily synthesized in large quantities in a single reaction vessel.

Translation of messenger RNA (mRNA) is a process in which the sequence of nucleotides in an mRNA molecule directs the incorporation of amino acids into protein. Translation of mRNA into protein is performed by a ribosome (ribosome-directed translation) and depends on transfer RNA (tRNA) molecules. tRNA molecules recognize and bind both to a codon in the mRNA and, at another site on the tRNA, to a particular amino acid. Each ribosome has three binding sites for tRNA: the aminoacyl tRNA site (the A site), the peptidyl-tRNA site (the P site) and the exit site (the E site).

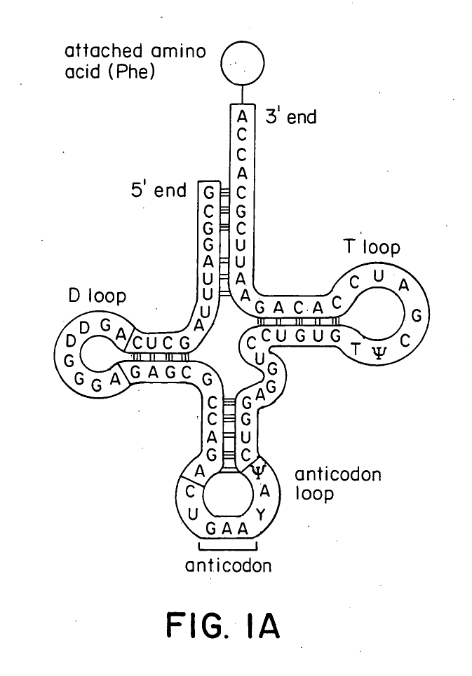

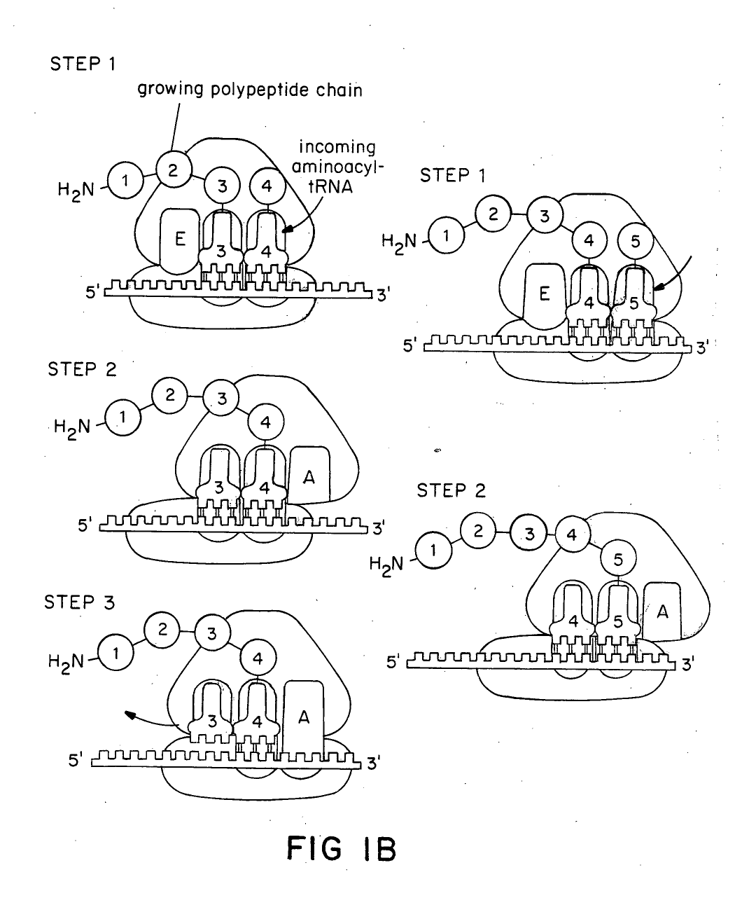

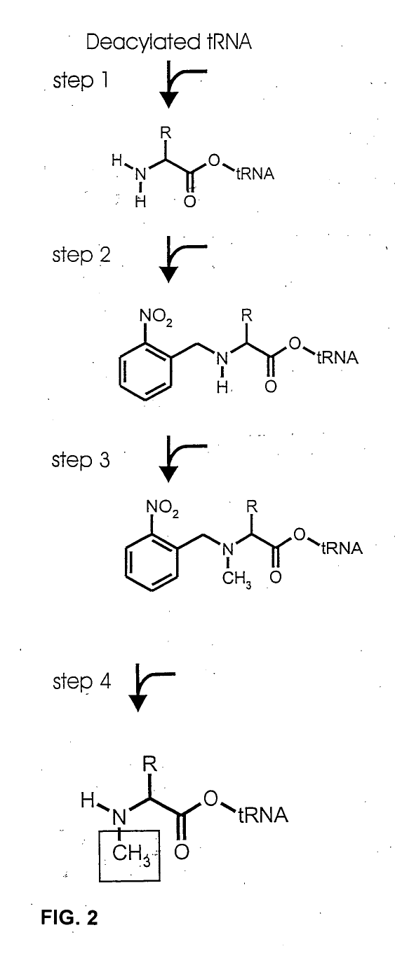

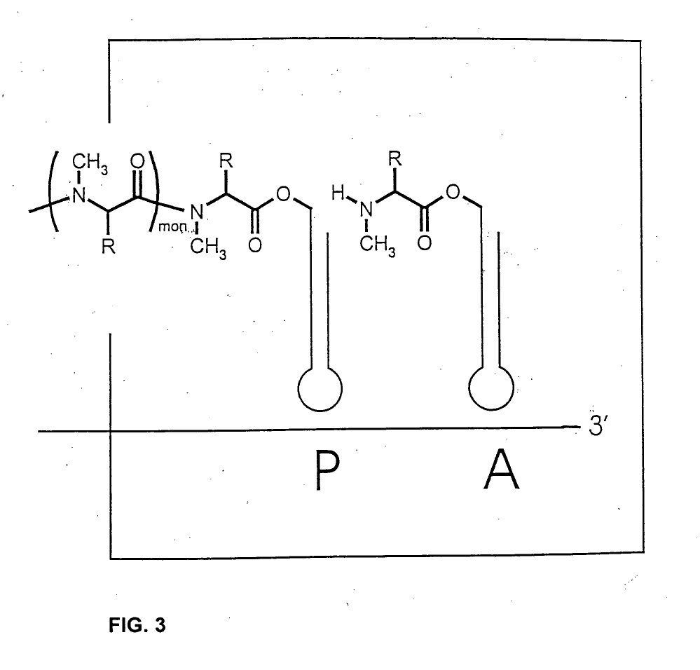

A tRNA molecule contains four short segments of nucleic acid that are double helical, and is often represented as a cloverleaf structure when drawn schematically (FIG. 1A). Two regions of unpaired nucleotides situated at either end of the tRNA molecule are crucial to the function of tRNA in protein synthesis. One of these regions forms the anticodon, a set of three consecutive nucleotides that pairs with the complementary codon (a set of three nucleotides) in an mRNA molecule, and the other is a short single stranded region at the 3′ end of the tRNA which is the site where the amino acid that matches the codon is attached to the tRNA (see FIG. 1A). Enzymes known as aminoacyl-tRNA synthetases covalently couple each amino acid to its appropriate tRNA molecule, thereby producing an aminoacyl tRNA (FIG. 1A).

During ribosome-directed translation, amino acids are added to the C-terminal end of a growing polymer (e.g, a growing polypeptide chain) during translation of mRNA into protein. Each amino acid is selected by complementary base pairing between a codon on the mRNA chain and the anticodon on its attached tRNA molecule. Because only specific tRNA molecules can base pair with each codon, the codon determines the specific amino acid to be added to the growing polypeptide chain. FIG. 1B shows the elongation process of a growing polypeptide chain in which a tRNA carrying the growing polypeptide is positioned in the P site of the ribosome. A tRNA carrying the next amino acid that is to be added to the chain, binds to the ribosomal A site by base pairing with the codon in an mRNA molecule, thereby acting as an acceptor substrate. The P site and the A site contain adjacent bound tRNAs (step 1). The carboxyl end of the polypeptide chain is released from the tRNA at the P site (by breakage of the high energy bond between tRNA and its amino acid) and joined to the single amino acid linked to the tRNA at the A site, forming a new peptide bond (step 2). The P-site tRNA thus also acts as a donor substrate in ribosome directed translation. This reaction is catalyzed by a peptidyl transferase catalytic activity contained in the ribosome and is accompanied by conformational changes in the ribosome which shift the two tRNAs into the E and P sites, respectively. The mRNA also moves three nucleotides through the ribosome and resets the ribosome so it is ready to receive the next aminoacyl tRNA in the unoccupied A site (step 3). Steps 1-3 are then repeated with the next incoming aminoacyl tRNA and so on (Alberts et al., Molecular Biology Of the Cell, 4^{th} ed. (2002)).

The ribosome-based synthesis of non-standard polymers was demonstrated over 30 years ago and, as expected, non-standard polymers can also be encoded with mRNA (Merryman, C., et al., Chem. & Biol., 9:741-746 (2002)). Nevertheless, the significant challenge that remains before evolutionary methods can be employed to select novel molecules is the generation of diverse and chemically promising starting population. To achieve high diversity with low molecular weight libraries that are suitable for drug discovery (M.W. ˜1000; chain length ˜10), about 20 different building blocks (acylated tRNAs) will be required (10¹³˜20¹⁰). The next task then is to generate 20 or so non-standard aminoacyl-tRNAs in sufficient quantity for successive rounds of ribosome-based encoding. Because mRNA encoding systems produce about 10¹¹ to 10¹² library members per ml, a diverse library of 10¹³ would require a translation reaction as large as 100 ml. At normal in vitro protein synthesis concentrations (1 mg/ml tRNA), a considerable amount of tRNA would be necessary (10-100 mg). To further complicate matters, current chemical misacylation techniques depend on individually isolated tRNAs, separate reactions, multiple steps, and commercially unavailable reagents (Noren, C. J., et al., Science, 244:182-188 (1989); Bain, J. D., et al., Tetrahedron, 47:2389-2400 (1991); Mendel, D., et al., J. Am. Chem. Soc., 115:4359-4360 (1993); Thorson, J. S., et al., Meth. Mol. Biol., 77:43073 (1998); Hohsaka, T., et al., Am. Chem. Soc., 121:34-40 (1999)).

An alternative to chemical misacylation is provided by the methods of the present invention. As described herein, nineteen of the standard twenty amino acids (all except proline) are modified (transformed, substituted) after they have been attached to their respective tRNAs by aminoacyl tRNA synthetases. As a result, readily available and inexpensive bulk tRNA can be used with the methods of the present invention because the synthetases ensure that the specific amino acids are attached to specific tRNAs. Subsequent chemical transformation of the whole population of tRNA-bound amino acids then allows for the immediate synthesis of 19 altered monomers (modified or substituted amino acids) that are ready for translation and addition to a growing polypeptide chain.

In one embodiment, the present invention relates to a method for producing an aminoacyl tRNA analogue comprising a tRNA and a modified amino acid which acts as an acceptor substrate and a donor substrate for ribosome-directed translation comprising substituting an aminoacyl tRNA under conditions in which an α-amino group of the aminoacyl group is monosubstituted with a group other than H, such that the aminoacyl tRNA analogue acts as an acceptor substrate and a donor substrate for ribosome-directed translation, thereby producing the aminoacyl tRNA analogue.

It is understood that as used herein the production of “an aminoacyl tRNA analogue” also includes the production of one or more aminoacyl tRNA analogues.

In another embodiment, the invention relates to a method for producing an aminoacyl tRNA analogue comprising combining a tRNA, an amino acid, and an aminoacyl tRNA synthetase under conditions in which an aminoacyl tRNA is formed. The aminoacyl tRNA is substituted under conditions in which an α-amino group of the aminoacyl tRNA is monosubstituted with a group other than H, such that the aminoacyl tRNA analogue acts as an acceptor substrate and a donor substrate for ribosome-directed translation, thereby producing the aminoacyl tRNA analogue.

In a particular embodiment, the present invention relates to a method of producing an aminoacylated tRNA analogue represented by the following structural formula: 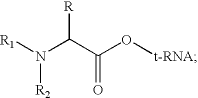
wherein:
R is an amino acid side chain;
R₁ is H or an amine protecting group;
R₂ is a group other than H such that the compound represented by the following formula acts as an acceptor substrate and a donor substrate for ribosome-directed translation: 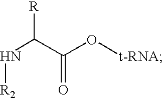
said method comprising the step of reacting a starting compound with a reagent capable of converting R₁—NH—R″ to R₁—NR₂—R″, wherein R″ is an organic moiety, under conditions suitable for said reaction, wherein:
the starting compound is represented by the following structural formula: 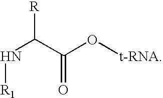
   An “organic moiety” as used herein refers, to a carbon containing compound. In one embodiment, the organic moiety is the remaining portion of the aminoacyl tRNA molecule.

The aminoacyl tRNA analogue of the present invention comprises a tRNA and a modified amino acid in which an α-amino group of the aminoacyl group is monosubstituted and wherein the tRNA analouge acts as an acceptor substrate and a donor substrate for ribosome-directed translation.

The tRNA can be any tRNA or tRNA-like molecule and can be produced or obtained from commercial sources (Sigma, Subriden). Suitable tRNAs include, for example, tRNA from any prokaryotic or eukaryotic organism, such as bacterial tRNA, yeast tRNA or mammalian tRNA, and altered versions thereof, so long as the tRNA can be charged with an amino acid or amino acid-like molecule and used in ribosome-directed translation. The tRNA can also be tRNA-like molecules or synthetic tRNAs, including T7 transcripts, as described, for example, by Hall, K. B., et al., Biochemistry 28:5794-5801 (1989). The tRNA can also be tRNA-like molecules comprising DNA as described, for example, by Khan and Roe, Science 241:74-79 (1988), so long as the tRNA-like molecule can be charged with an amino acid or amino acid-like molecule and used in ribosome-directed translation.

The amino acid of the aminoacyl tRNA can be derived from any naturally occurring amino acid or analogue thereof, so long as when the amino acid or its analogue is part of the tRNA analogue, the resulting aminoacyl tRNA analogue can act as an acceptor substrate and a donor substrate for ribosome-directed translation. The amino acid can be, for example, alanine, arginine, asparagine, aspartic acid, cysteine, glutamine, glutamic acid, glycine, histidine, isoleucine, lysine, methionine, phenylalanine, serine, threonine, tryptophan, tyrosine, valine, and analogues thereof. Amino acids analogues are well known in the art. Suitable amino acid analogues that can be incorporated into protein include, for example, those listed in Horton and Boime, Methods in Enzymology 96:777 (1983).

One of ordinary skill in the art would be able to determine which aminoacyl tRNA analogues produced by the methods of the present invention “act as donor and acceptor substrates for ribosome directed translation” using assays that are well known in the art. For example, a given aminoacyl tRNA analogue can be detectably labeled and used to synthesize dipeptides as described in the Examples. Alternatively, the aminoacyl tRNA analogue can be detectably labeled and used in the translation of a suitable mRNA, where the products of the translation are precipitated, and the amount of label in the precipitate is indicative of the level of incorporation of the aminoacyl tRNA analogue into a polymer.

As used herein, an aminoacylated tRNA analogue that acts as an acceptor substrate and donor substrate for ribosome-directed translation is one that can be recruited to the A-site of a ribosome during ribosome-directed translation, such that the nascent polymer (present in the P-site) can be transferred to the modified amino acid portion of the aminoacyl tRNA analogue. In addition, when the aminoacyl tRNA analogue is in the P-site, the nascent polymer can be transfered to the amino acid of the A-site tRNA, thereby acting as an acceptor substrate and a donor substrate for ribosome-directed translation.

Aminoacyl tRNA can be produced by methods well known in the art, including in vitro methods using bacterial cell extracts and tRNA synthetases. The examples provide one way of producing aminoacylated tRNA. Numerous methods for producing aminoacyl tRNA are available. One of ordinary skill in the art would be able to select a suitable method. Furthermore, suitable aminoacyl tRNAs are commercially available. For example, Transend™ is a tRNA charged with biotinylated leucine and can be obtained from Promega.

Monosubstitution of an α-amino group of the aminoacyl tRNA refers to an aminoacyl tRNA analogue having the following structural formula: 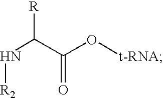
wherein R₂ is a organic moiety or group other than H such that the aminoaclyated tRNA analogue acts as an acceptor substrate and a donor substrate for ribosome-directed translation. That is, only one H of the amine group (NH₂) is substituted and the presence of the R₂ group in the aminoacyl tRNA analogue does not substantially interfere with ribosome directed translation. “Interfering with ribosome directed translation” refers to affecting the ability of the ribosome to produce polymers to such a degree that the polymers are not usable for in vitro selection as described herein. One of ordinary skill in the art would be able to use suitable selection and amplification techniques to detect, and/or identify, and/or evolve a non-standard polymer of interest, even where the library produced is small or the non-standard polymer of interest is a rare member of the library.

In one embodiment, the R₂ group is of a size (large size) such that when contacted with an aminoacyl tRNA, R₂ sterically hinders occurrence of disubstitution (e.g., ethyl, propyl, and secpropyl groups). Additional R2 groups can be derived from the aldehydes and ketones described in Ohfune, et al., Chem. Let. 3:441-444 (1984).

In another embodiment, the R₂ group is of a size (smaller size) such that when contacted with an aminoacyl tRNA, R₂ does not sterically hinder the occurrence of disubstitution (e.g., methyl group). That is, disubstitution of the amine group would occur. Therefore, in this embodiment, the amine group of the tRNA analogue is contacted with a protecting group prior to or simultaneously with an R₂ group in order to prevent disubstitution from occurring, thereby producing a protected aminoacyl tRNA. After substitution of the α-amino group of the aminoacyl tRNA (after contacting the protected aminoacyl tRNA an R₂ group), the protecting group can be removed, thereby deprotecting the aminoacyl tRNA analogue.

Protecting groups for amines and conditions for protecting and deprotecting amines with these protecting groups are well known in art and are disclosed, for example in T. W. Greene & P. G. M. Wuts, Protective Groups in Organic Synthesis, 2^{nd} ed., J. Wiley & Sons (1991) and P. J. Kocienski, Protecting Groups, George Thieme Verlag (1994). Examples of protection groups for amines include carbamates (e.g., 9-Fluorenylmethyl carbamate (Fmoc); 2,2,2-Trichloroethyl carbamate; 2-Trimethylsilylethyl Carbamate (Teoc); t-Butyl Carbamate (BOC); allyl carbamate; benzyl carbamate; m-Nitrophenyl carbamate), amides (e.g., formamides, acetamides, trifluroracetamides), and sulfonamides (e.g., p-Toluenesulfonyl, trifluoromethanesulfonyl, trimethylsilylethanesulfonamide, tert-Butylsulfonyl (Bus)). In a particular embodiment, a protected aminoacyl tRNA is produced by contacting the tRNA with an o-nitrobenzaldehyde and sodium cyanoborohydride.

Examples of suitable protecting groups include those that can reductively aminate the α-amino group of an amino acid attached to a tRNA under mild, aqueous conditions, and then removed under mild aqueous conditions. For example, one of ordinary skill in the art would be able to select suitable substituted nitrobenzaldehydes. In this embodiment, the resulting protected aminoacyl tRNA comprises an aminoacyl α-amino group monosubstituted with a nitrophenyl group. In a particular embodiment, the aminoacyl tRNA is contacted with o-nitrobenzaldehyde, and the resulting protected aminoacyl tRNA is monosubstituted with an o-nitrophenyl group. Another example of a suitable protecting group is NVOC-C1 (nitroveratrylcarbonyl (Fluka, Aldrich).

Where the aminoacyl tRNA has been protected and then further substituted, producing a disubstituted aminoacylated tRNA, the disubstituted aminoacylated tRNA is then deprotected to remove the protecting group. In one embodiment, the disubstituted aminoacylated tRNA is deprotected by photoreversal, resulting in an aminoacyl tRNA analogue comprising a tRNA and a modified amino acid. In a particular embodiment, the disubstituted tRNA is deprotected by exposing it to UV irradiation for 10 minutes (at a distance of 10 cm) with a 550 watt, mercury vapor lamp (ACE glass).

One of ordinary skill in the art would be able to select other suitable protecting groups and methods for removing the protection group (deprotecting).

Reactions with amines, such as the α-amino group of an aminoacylated tRNA (free amines or protected amines) are well known in the art and are disclosed for example in March, J., Advanced Organic Chemistry, 4^{th} ed., John Wiley & Sons (1992). Such reactions include, for example, alkylation reactions, sulfonylation reactions, acylation reactions and reductive amination reactions.

In a particular embodiment, the aminoacyl tRNA is contacted with a lower alkyl group containing molecule, under conditions in which the α-amino group of the aminoacylated tRNA is monosubstituted with the alky group. Such a group can be attached to the aminoacyl tRNA using a reductive alkylation reaction. The aminoacyl tRNA can be modified under mild, aqueous conditions in which the ability of the resulting charged tRNA to be used in ribosome-directed translation is retained.

Where the alky group is of sufficient size, steric hindrance allows only monosubstitution of the α-amino group, thereby producing an aminoacyl tRNA analogue comprising a tRNA and a modified amino acid wherein α-amino group of the aminoacyl tRNA is monosubstituted with a group other than H, such that the aminoacyl tRNA analogue acts as an acceptor substrate and a donor substrate for ribosome-directed translation, thereby producing the aminoacyl tRNA analogue. In a particular embodiment, the alky group contains two or more carbons.

Suitable lower alkyl containing molecules include aldehydes of the structure 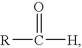
wherein R can be, for example, H, CH₃, and CH₃(CH₂) In one embodiment, the aldehyde is formaldehyde. Suitable lower alkyl containing molecules also include ketones of the structure 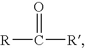
wherein R can be, for example, CH₃, and CH₃(CH₂), and R′ can be, for example, CH₃, and CH₃(CH). In a particular embodiment, the ketone is acetone. The aminoacyl tRNA is contacted with a lower alky group containing molecule at a concentration suitable to produce an aminoacyl tRNA analogue wherein the α-amino group of the amino acid is monosubstituted with the lower alkyl group. While not wishing to be bound by theory, low concentrations of alky group containing compound can be used relative to the concentration of tRNA. The lower the concentration of alkyl group containing compound, the slower the reaction will proceed. Furthermore, the aminoacyl tRNA has a half life. Therefore, the longer the reaction takes, the more aminoacyl tRNAs will lose the amino acid. In one embodiment, the lower alkyl group containing compound is present at a concentration in excess over the amount of tRNA. Having the alkyl group containing compound present in excess over the concentration of tRNA is expected to increase the rate of the reaction and drive it toward completion, where all of the aminoacylated tRNAs are modified. One of ordinary skill in the art would be able to select a suitable concentration of alkyl group containing compound in relation to tRNA in order to produce the aminoacyl tRNA analogues of the present invention and reduce or minimize the lose of aminoacyl tRNA if desired.

Thus, in one embodiment of the method of producing an aminoacyl tRNA analogue, substituting the aminoacyl tRNA under conditions in which the α-amino group of the aminoacylated tRNA is monosubstituted comprises a) protecting the α-amino group of the aminoacyl group wherein one H of the NH₂ group is substituted with a protecting group, b) substituting the H of the NH₂ group, and then c) removing the protecting group (deprotecting), thereby producing an aminoacyl tRNA analogue comprising a tRNA and a modified amino acid which acts as an acceptor substrate and a donor substrate for ribosome-directed translation. The methods of the present invention can be conducted under mild, aqueous conditions.

In a particular embodiment, the aminoacylated tRNA is contacted with o-nitrobenzaldehyde or a substituted o-nitrobenzaldehyde under conditions in which one H of the α-amino group of the aminoacyl group is substituted with a nitrophenyl group or a substituted nitrophenyl group, thereby producing a protected aminoacylated tRNA. The protected aminoacylated tRNA is contacted with a suitable alky-containing compound under conditions in which the other H of the α-amino group of the aminoacyl group is substituted, thereby producing a disubstituted aminoacyl tRNA. The disubstituted aminoacylated tRNA is deprotected, thereby producing an aminoacyl tRNA analogue comprising a tRNA and a modified amino acid wherein the cl-amino group of the aminoacyl tRNA is monosubstituted and the aminoacyl tRNA analogue acts as an acceptor substrate and a donor substrate for ribosome-directed translation. Mild aqueous conditions suitable for the protection reaction are as described above for the substitution and in the Examples. One of ordinary skill in the art would be able to vary the concentrations of the aminoacyl tRNA, the protecting regent, and the reducing agent (e.g. an imine reducing agent such as sodium cyanoborohydride) in order to substitute the aminoacyl tRNA with a protecting group.

The method for producing an aminoacyl tRNA analogue comprising a tRNA and a modified amino acid wherein the aminoacyl tRNA analogue acts as an acceptor substrate and a donor substrate for ribosome-directed translation can also include the step of coupling (also referred to herein as “charging”) the tRNA with the appropriate amino acid, resulting in aminoacyl tRNA. In one embodiment, prior to substituting the aminoacyl tRNA under conditions in which an α-amino group of the aminoacylated tRNA is monosubstituted, tRNA, amino acid, and aminoacyl tRNA synthetase are combined under conditions in which aminoacylated tRNA is formed. (Merryman, et al., Chem. & Biol. 9:741-746 (2002); and Merryman and Green, Chem & Biol. Online publication Mar. 18, 2004 DOI: 10.1016/S1074552104000857 on the world wide web at chembiol.com/content/future). In a specific embodiment, total tRNA (a mixture of all tRNAs specific for each of the 20 natural amino acids) is contacted with a crude extract containing tRNA synthetases, thereby producing a mixture of aminoacyl tRNAs. tRNA and aminoacyl tRNA synthetases can be obtained by methods well known in the art.

In one embodiment, the method comprises combining a plurality of tRNAs that are specific for a plurality of amino acids, amino acids which are specific for a subset of the tRNAs, and aminoacyl tRNA synthetases, under conditions in which the subset of tRNAs are aminoacylated, thereby forming a mixture of tRNAs that are aminoacylated and tRNAs that are not aminoacylated. The tRNAs that are aminoacylated are substituted under conditions in which the α-amino group of each aminoacylated tRNA is monosubstituted with a group other than H, thereby forming aminoacyl tRNA analogues which act as acceptor substrates and donor substrates for ribosome-directed translation. The mixture is then contacted with amino acids that are specific for the tRNAs that are not aminoacylated, under conditions in which aminoacylated tRNAs are formed, thereby producing the population of tRNAs comprising aminoacyl tRNAs and aminoacyl tRNA analogues.

The invention is also drawn to a method for producing a population of tRNA comprising at least one aminoacyl tRNA and at least one aminoacyl tRNA analogue, wherein each tRNA analogue comprises a tRNA and a modified amino acid which acts as an acceptor substrate and a donor substrate for ribosome-directed translation.

In one embodiment, the method comprises combining a plurality of tRNAs that are specific for a plurality of amino acids, amino acids which are specific for a subset of the tRNAs, and aminoacyl tRNA synthetases, under conditions in which the subset of tRNAs are aminoacylated, thereby forming a mixture of tRNAs that are aminoacylated and tRNAs that are not aminoacylated. The tRNAs that are aminoacylated are substituted under conditions in which the α-amino group of each aminoacylated tRNA is monosubstituted with a group other than H, thereby forming aminoacyl tRNA analogues which act as acceptor substrates and donor substrates for ribosome-directed translation. The mixture is then contacted with amino acids that are specific for the tRNAs that are not aminoacylated, under conditions in which aminoacylated tRNAs are formed, thereby producing the population of tRNAs comprising aminoacyl tRNAs and aminoacyl tRNA analogues.

In another embodiment, a crude population of tRNAs is separated into two sets using methods known in the art. Suitable methods for separating the tRNAs are described, for example, by Ribeiro, et al., Analytical Biochemistry 228:330 (1995); Johnson, B., Biochemistry 20:6103 (1981); Goss and Parkhurst, J. Biol. Chem. 253:7804 (1978); and Wang and Wong, Anal. Biochem. 131:360-364 (1983). The first set of tRNA is contacted with amino acids and/or amino acid analogues, and aminoacyl tRNA synthetases under conditions in which the tRNA are aminoacylated with the appropriate amino and/or amino acid analogue. The amino acids can include those wherein the corresponding tRNA is not present in the tRNA set. The aminoacyl tRNA is then monosubstituted as described above; thereby producing at least one aminoacyl tRNA analogue comprising a tRNA and a modified amino acid which acts as an acceptor substrate and a donor substrate for ribosome-directed translation. The second set of tRNAs is contacted with amino acids and/or amino acid analogues, and tRNA synthetases under conditions in which the tRNA are aminoacylated with the appropriate amino acid or amino acid analogue. The aminoacyl tRNA analogues can be mixed with the aminoacyl tRNAs and used to produce the non-standard polymers of the present invention.

In an alternate embodiment, a crude population of tRNA is contacted with tRNA synthetases and amino acids and/or amino acid analogues, wherein the amino acids and/or amino acid analogues correspond to a subset of the tRNAs present, under conditions in which the tRNAs are aminoacylated with the appropriate amino acid or amino acid analogue, if present, resulting in a population of tRNA and aminoacylated tRNA. The population of tRNA and aminoacylated tRNA is then substituted, as described above, producing a population of tRNA and aminoacyl tRNA analogues comprising a tRNA and a modified amino acid which acts as an acceptor substrate and donor substrate for ribosome-directed translation. Amino acids and/or amino acid analogues corresponding to those that were absent in the first acylation reaction are then added to the population of tRNA and aminoacyl tRNA analogues under conditions in which the tRNA are aminoacylated, thereby producing a population of tRNA comprising at least one aminoacyl tRNA analogue and at least one aminoacyl tRNA. The population of aminoacyl tRNA analogues and aminoacyl tRNA can then be used to produce non-standard polymers comprising a mixture of amino acids and modified amino acids and/or analogues thereof by ribosome-directed translation.

The present invention is also drawn to methods for producing a non-standard polymer. The method comprises producing an aminoacyl tRNA analogue comprising substituting an aminoacyl tRNA under conditions in which the α-amino group of the aminoacylated tRNA is monosubstituted is monosubstituted with a group other than H, such that the aminoacyl tRNA analogue acts as an acceptor substrate and a donor substrate for ribosome-directed translation. The aminoacyl tRNA analogue is combined with an encoding nucleic acid, and an in vitro translation mixture. The in vitro translation mixture can be, for example, a bacterial translation mixture, such as from E. coli; eukaryotic translation mixtures, such as mixtures from wheat germ or rabbit reticulocytes, or translation mixtures from other organisms. It is understood that tRNA isolated from one organism can be used in combination with an in vitro translation system from a different organism. The combination is maintained under conditions in which the aminoacyl tRNA analogue is used in the translation of the encoding nucleic acid, thereby producing a non-standard polymer. The aminoacyl tRNA analogue can also be produced separately and combined with the encoding nucleic acid and in vitro translation mixture as described above.

The encoding nucleic acid can be any mRNA or mRNA-like molecule (of any sequence or length) that is translated by the ribosome. The encoding nucleic acid contains elements required for its translation. The encoding nucleic acid can also include elements, such as primer binding sites, that permit it to be reverse transcribed (if necessary) and amplified. The encoding nucleic acid can also include elements, such as suitable restriction sites, that permit it to be cloned. In another embodiment, the primers used to reverse transcribe or amplify the encoding nucleic acid can include additional sequence that contains suitable restriction sites.

In one embodiment of the method for producing a non-standard polymer, the method further comprises forming a stable complex that includes the non-standard polymer and its encoding nucleic acid, such that the non-standard polymer and its encoding nucleic acid can be co-isolated. The stable complexes are formed such that non-standard polymer and its encoding nucleic acid can be co-isolated wherein the encoding nucleic acid can be reversed transcribed, if necessary, and/or amplified, and/or cloned.

In one embodiment for forming a stable complex, the in vitro translation mixture includes tRNA having a Y base and the Y base is activated to covalently crosslink the Y base containing tRNA to the encoding nucleic acid, thereby forming the stable complex.

In another embodiment, stable complexes are formed by cross linking the encoding nucleic acid to a bifunctional tRNA as described in U.S. Pat. No. 6,440,695. In another embodiment, stable complexes are formed as described by Gold, U.S. Pat. 5,843,701

In still another embodiment, stable complexes are formed by mRNA display. As described in published patent application, US 2003/023581 A1, the encoding nucleic acid modified with a “peptide acceptor” at the 3′ terminus of the encoding nucleic acid. The peptide acceptor can be, for example, puromycin, which acts to attach a growing peptide to its encoding mRNA as descried in U.S. Pat. No. 6,281,344. Other suitable peptide acceptors are described in U.S. 2003/023581A1.

The present invention also relates to a diverse library or collection of encoded non-standard polymers synthesized in translation systems containing the tRNA analogues of the present invention. The method comprises producing an aminoacyl tRNA analogue comprising substituting an aminoacyl tRNA under conditions in which the α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue. The aminoacyl tRNA analogue is combined with a population of encoding nucleic acids and an in vitro translation mixture. The combination is maintained under conditions in which the aminoacyl tRNA analogue is used in the translation of the population of encoding nucleic acids, thereby producing a library comprising non-standard polymers. In one embodiment, the combination is maintained under conditions suitable to form stable complexes that include non-standard polymers and their encoding nucleic acids, such that a non-standard polymer having a desired property can be co-isolated with its encoding nucleic acid. Methods for forming stable complexes are described above. In one embodiment, substituting the aminoacyl tRNA under conditions in which an α-amino group of the aminoacylated tRNA is monosubstituted comprises protecting, substituting and deprotecting the aminoacyl α-amino group as described above. The aminoacyl tRNA analogue can also be produced separately and combined with the encoding nucleic acid and in vitro translation mixture as described above.

The present invention also relates to the library provided by the method.

The method of producing a non-standard polymer can comprise a number of different types of aminoacyl tRNA populations, thereby producing non-standard polymers having a range of modified amino acid content. For example, a single tRNA can be used in the substitution step, producing the corresponding aminoacyl tRNA analogue. In this embodiment, the remaining aminoacyl tRNA necessary for translation of the encoding nucleic acid are supplied without the modification described herein. In one embodiment, a mixed population of aminoacyl tRNA and aminoacyl tRNA analogues, produced as described above are used, resulting in non-standard polymers comprising a mixture of amino acids and/or amino acid analogues, and modified amino acids and/or amino acid analogues. In still another embodiment, a subset of aminoacyl tRNA analogues is produced and the in vitro translation mixture includes the remaining aminoacyl tRNA necessary to transcribe the encoding nucleic acid. In another embodiment of the method of producing a non-standard polymer, a complete set of aminoacyl tRNA representing each of the 20 natural amino acids is produced and subjected to modification as described above. Any combination of aminoacyl tRNA and aminoacyl tRNA analogues can be produced by the method of the present invention and used to produce non-standard polymers.

The present invention is also drawn to a method for identifying a non-standard polymer having a desired activity. The method comprises generating a library comprising non-standard polymers as described above. A member of the library that exhibits a desired activity is selected, thereby identifying a non-standard polymer having a desired activity. The aminoacyl tRNA analogue can also be produced separately, and combined with the encoding nucleic acid and in vitro translation mixture as described above.

In one embodiment, the combination of aminoacyl tRNA analogue, encoding nucleic acids and in vitro translation mixture is maintained under conditions suitable to form stable complexes that include non-standard polymers and their encoding nucleic acids, such that a non-standard polymer having a desired property and its encoding nucleic acid can be co-isolated as described above. The encoding nucleic acid of the selected library member is then amplified by methods well known in the art. The process of generating a library comprising non-standard polymers and selecting a member of the library that exhibits a desired activity can be repeated, using the amplified nucleic acid as the population of encoding nucleic acids in the combination with aminoacyl tRNA analogues and the in vitro translation mixture. A key advantage of the encoded non-standard polymer containing library of the present invention is that even members which occur in small numbers (rare members) and are of interest because of desired biological or biochemical properties (e.g., binding to a particular ligand, enzymatic activity) can be enriched and then identified by amplification, cloning and sequencing of their respective encoding nucleic acid.

A diverse library comprising encoded non-standard polymers can be enriched in molecules with the desired properties using known methods, to identify target members. Methods by which target members of the library can be enriched include affinity enrichment using immobilized ligand or binding partner and, for enzymatic activity, affinity to a product of a reaction in which the enzyme has modified itself (with, for example, a mechanism based inhibitor) or a substrate to which it is attached (e.g., Williams, K. P. and D. P. Bartel, “In Vitro Selection of Catalytic RNA”, pp. 367 381 In: Catalytic RNA, (Fritz Eckstein and David M. J. Lilley, Ed.), Springer, (1996)).

Furthermore, libraries enriched in target members can be amplified and subjected to additional enrichment. For example, where the encoding nucleic acid co-isolated with the encoded non-standard polymer is mRNA, it can be reverse transcribed, producing the cDNAs of the mRNA components. The cDNAs can then be amplified (e.g., by PCR or other amplification methods). The resulting PCR products are subjected to in vitro transcription, resulting in production of an amplified pool of mRNAs that encode the members of the enriched fusion library. In vitro translation of this pool in the presence of the tRNA analogues of the present invention, produces an amplified version of the enriched encoded non-standard polymer library. Library members selected and amplified in this way are subjected to further enrichment and amplification, which is repeated as necessary until target members are enriched to the desired extent (e.g., enriched to a level where they are present in sufficient numbers to be detected by binding to a ligand of interest or catalyzing a reaction of interest). After sufficient enrichment, encoding nucleic acids of target members are cloned and individual non-standard polymers can be synthesized and screened for the desired function. The translation product of the encoding nucleic acid or a fragment of the translation product can also be screened for activity without co-isolation with the encoding nucleic acid.

The translation products of the enriched encoding nucleic acid (the non-standard polymer component of the target members), such as non-standard polymers which display activities of interest (e.g., ligand binding or catalytic activity), as well as engineered derivatives of these translation products which display activities of interest, are referred to as targets. These targets are also the subject of this invention. Targets can be released or separated from the nucleic acids with which they were co-isolated, using known methods. They can be used, for example, as diagnostic or therapeutic reagents (e.g., single chain monoclonal antibodies),catalysts, members of binding pairs, ligands, enzyme substrates. Once a non-standard polymer fragment which has desired characteristics has been identified, it can be produced using known methods (e.g., production in an appropriate expression system, chemical synthesis).

The present invention is also drawn to tRNA analogues. In one embodiment, the tRNA analogues of the present invention comprise a tRNA and a modified amino acid wherein an α-amino group of the amino acid is monosubstituted with a lower alkyl group, and wherein if the α-amino group is monosubstituted with a methyl group, the amino acid is other than leucine, glycine, phenylalanine or alanine, wherein the aminoacyl tRNA analogue acts as an acceptor substrate and a donor substrate for ribosome-directed. In one embodiment, the lower alkyl group is a methyl group. The amino acid can be, for example, alanine, arginine, asparagine, aspartic acid, cysteine, glutamine, glutamic acid, glycine, histidine, isoleucine, lysine, methionine, phenylalanine, serine, threonine, tryptophan, tyrosine, valine, analogues thereof, and combinations of said amino acids and analogues thereof. In one embodiment, the mixture of aminoacyl tRNAs and aminoacyl tRNA analogues comprises aminoacyl tRNAs including the amino acids Glu, Lys, Asn, Arg and Trp and aminoacyl tRNA analogues including the amino acids alanine, aspartic acid, cysteine, glutamine, glycine, histidine, isoleucine, methionine, phenylalanine, serine, threonine, tyrosine, and valine. One of ordinary skill in the art can select the desired mixture of aminoacyl tRNAs and aminoacyl tRNA analogues.

The present invention is also drawn to a population of tRNA analogues wherein wherein each aminoacyl tRNA analogue comprises a tRNA and a modified amino acid wherein an α-amino group of each amino acid is monosubstituted with a lower alkyl group, and wherein the amino acids include alanine, arginine, asparagine, aspartic acid, cysteine, glutamine, glutamic acid, glycine, histidine, isoleucine, lysine, methionine, phenylalanine, serine, threonine, tryptophan, tyrosine, and valine, wherein the aminoacyl tRNA analogues act as acceptor substrates and donor substrates for ribosome-directed translation.

The present invention is also drawn to an aminoacyl tRNA analogue comprising a tRNA and modified amino acid wherein the α-amino group of the amino acid is substituted with a protecting group. tRNA analogues comprising a modified amino acid wherein the α-amino group of the amino acid is substituted with a protecting group are stable and can be used to produce tRNA analogues comprising a tRNA and a modified amino acid which acts as an acceptor substrate and a donor substrate for ribosome-directed translation, wherein an α-amino group of the amino acid is monosubstituted with a lower alkyl group, as described herein. The protecting group can be o-nitrophenyl and substituted versions thereof. The amino acid can be alanine, arginine, asparagine, aspartic acid, cysteine, glutamine, glutamic acid, glycine, histidine, isoleucine, lysine, methionine, phenylalanine, serine, threonine, tryptophan, tyrosine, valine, analogues thereof, and combinations of said amino acids and analogues thereof.

The present invention is also drawn to an aminoacyl tRNA analogue or plurality of aminoacyl tRNA analogues wherein each tRNA analogue comprises a tRNA and a modified amino acid, wherein the α-amino group of the amino acid is monosubstituted with a protecting group, and wherein the amino acids include alanine, arginine, asparagine, aspartic acid, cysteine, glutamine, glutamic acid, glycine, histidine, isoleucine, lysine, methionine, phenylalanine, serine, threonine, tryptophan, tyrosine, and valine, analogues thereof, and combinations of said amino acids and analogues thereof.

The present invention is also drawn to a tRNA analogue or plurality of aminoacyl tRNA analogues comprising a tRNA and a modified amino acid attached to the tRNA such that it can act as an acceptor and a donor substrate for ribosome-directed translation, wherein an α-amino group of the amino acid is substituted with a protecting group and with a lower alkyl group. tRNA analogues comprising a modified amino acid wherein the α-amino group of the amino acid is substituted with a protecting group and a lower alky group are stable when kept in the dark and can be used to produce tRNA analogues comprising a tRNA and a modified amino acid which acts as an acceptor substrate and a donor substrate for ribosome-directed translation, wherein the α-amino group of the amino acid is monosubstituted with a lower alkyl group as described herein. In a particular embodiment, the lower alkyl group is a methyl group. The amino acid can be alanine, arginine, asparagine, aspartic acid, cysteine, glutamine, glutamic acid, glycine, histidine, isoleucine, lysine, methionine, phenylalanine, serine, threonine, tryptophan, tyrosine, valine, analogues thereof and combinations of said amino acids and analogues thereof. In one embodiment the plurality of aminoacyl tRNA analogues includes each of the amino acids listed above. The protecting group can be o-nitrophenyl and substituted versions thereof. In a particular embodiment, the protecting group is an o-nitrophenyl group. In another particular embodiment, the amino acid is selected from the group consisting of glutamic acid, serine, and valine.

In one embodiment, the tRNA analogue or plurality thereof is made according to the method of the present invention. In a particular embodiment, the tRNA analogue or plurality thereof is made simultaneously, in one tube or container.

Ribosomes can polymerize well over 100 different amino acid analogs (Noren, C. J., et al., Science, 244:182-188 (1989); Bain, J. D., et al., Tetrahedron, 47:2389-2400 (1991); Mendel, D., et al., J. Am. Chem. Soc., 115:4359-4360 (1993); Thorson, J. S., et al., Meth. Mol. Biol., 77:43073 (1998); Hohsaka, T., et al., Am. Chem. Soc., 121:34-40 (1999)). As might be predicted from surveying the standard 20 amino acids, a wide variety of sidechains are acceptable. Perhaps of greater importance is the fact that unusual backbones can also be synthesized by the ribosome. Currently, the list of potential translation products includes D-polypeptides, polyesters, thioesters, thioamides, phosphinoamides, β-peptides, N-methyl polypeptides, polypeptides with quanternary α-carbons, hydrazine based polymers, and aminoocy based polymers (Calendar, R., et al., J. Mol. Biol., 26:39-54 (1967); Fahnestock, S. et al., Science, 173:340-343 (1971); Gooch, J., et al., Biocehm. J., 149:209-220 (1975); Victorova, L. S., et al., FEBS Letters, 68:215-218 (1976); Tarussova, N. B., et al., FEBS Letters, 130:85-87 n(1981); Ellman, J. A., et al., J. Am. Chem. Soc., 120:3032-3042 (1992); Eisenhauer, B. M., et al., /Biocehm., 41:11472-11478 (2002)). Using the methods of the present invention, ribosomes could be used to synthesize libraries with useful physical, chemical, and biological properties by translating randomized pools of mRNA in the presence of the aminoacyl tRNA analogues of the present invention. For example, poly-N-methyl peptides (methylated peptides) seem to be both protease resistant and to have the ability to cross cell membranes (Pohl, J., Pept.: Chem., Struct Biol., Proc. Am. Pept. Symp., 11^{th} Rivier, J. E. and Marshall, G. R. (Eds.) 365-366; Gordon, D. J., et al., Biochem., 40:8237-8245 (2001); Haviv, F., et al., J. Med. Chem., 36:363-369 (2001); Conradi, R. A., et al., Pharm. Res., 9:435-439 (1992); Chikhale, E. G., et al. Pharm. Res., 11:412-419 (1994); Sheehy, B. A., et al., Mol. And Biochem, Parasitol., 105:39-49 (2000)), making them an attractive class of compounds for drug discovery.

By itself, the ribosomal synthesis of non-standard polymers is an interesting but rather involved way of generating libraries that could be made easily by other approaches. However, ribosome synthesis is critical because it allows for the encoding of products with mRNA and the subsequent processing of staggeringly complex libraries (>10¹³) by in vitro selection (Mattheakis, L. C., et al., Proc. Natl. Acad. Sci. USA, 91:9022-9026 (1994); Nemoto, N., et al., FEBS Letters, 414:405-408 (1997); Roberts, R. W., et al., Proc. Natl. Acad. Sci. USA, 94:12297-12302 (1997); Merryman, C., et al., Chem. Biol., 9:741-746 (2002); Baskerville, S., et al., Proc. Natl. Acad. Sci. USA, 99:741-746 (2002); Keefe, A. D., et al., Nature, 410:715-718 (2001)). The unique power of evolutionary methods is that they make it comparatively simple to identify which library members have the desired function. The basic procedure is to use iterative rounds of selection and amplification, where the selective step increases the proportion of functional molecules, and amplification increases their number. With each round, the library is exponentially enriched in molecules that satisfy the selective criteria. Thus, an originally diverse population that may contain only a single copy of a desirable molecule quickly evolves into a population dominated by the molecule.

The transformation of aminoacyl tRNA as described herein is expected to allow production of polymers with useful properties. In addition, the chemical manipulations proceed in slightly acidic, aqueous media, which is important because the aminoacyl ester bond is base-labile and tRNA is insoluble in common organic solvents. Furthermore, using the present method, any side reactions apparently do not inactivate the 25 kDa tRNAs to which the amino acids are attached, allowing the aminoacylated tRNA analogues of the present invention to be used in ribosome-directed translation. Nearly quantitative yields are obtained, allowing on-to-one correspondence between the monomer borne by a tRNA and the codon the tRNA recognizes.

As shown herein, reductive alkylation with o-nitrobenzaldehyde followed by reductive methylation with formaldehyde and photoreversal of the initial adduct produces tRNAs bearing N-monomethyl amino acids. This simple transformation is quantitative and most of the N-methyl aminoacyl tRNAs are substrates for the ribosome. Because this approach can be used with bulk aminoacylated tRNA, it is trivial to simultaneously synthesize large amounts of numerous N-methly aminoacyl tRNAs. By eliminating the substantial synthetic efforts demanded by traditional methods, our approach provides a readily accessible means for producing and searching vast libraries (˜10¹³) of “drug-like” methylated peptides via ribosome-based encoding and in vitro selection.

The present invention is illustrated by the following examples, which are not intended to be limiting in any way.

### EXAMPLES

### Example 1

### Synthesis of Me-N-tRNAs by on tRNA Transformation

Preparation of S1 00 Extract

Twenty grams of E. coli cells were lysed and the cellular debris removed as described in Merryman, et al., Chem. & Biol. 9:741-746 (2002). Ribosomes were removed from the clarified cell lysate by centrifugation for 4 hours, at 4° C., at 40,000 rpm in a Beckman Ti60 rotor and the supernatant was diluted two fold with buffer D (10 mM Tris-HCl pH 7.5; 30 mM NH₄Cl; 10 mM MgCl₂; and 6 mM 2-mercaptoethanol (BME)). This solution was stirred with 12 g of dry DEAE-cellulose that was equilibrated with buffer D, washed with distilled water, and then dried. The slurry was filtered on a scintered glass funnel and washed with 1-2 liters of buffer D. The DEAE-cellulose cake was resuspended in buffer D and packed in a column. The S100 extract was then eluted with buffer D containing 250 mM NH₄Cl (the desired fractions elute as a sharp band and can usually be identified by eye as they have a pale yellow color). Small aliquots (100 μl) of the purified S100 extract were frozen in liquid nitrogen and stored at −70° C. The tRNA charging efficiency of S100 was determined by monitoring TCA-precipitable counts in the presence of tRNA^{phe} and radiolabeled phenylalanine [S100 is incubated for 10 minutes at 37° C. with tRNA^{phe} (1 μM), ¹⁴C-phenylalanine (20 μM), and ATP (2 mM) in 30 mM Tris-HCl pH 7.5, 15 mM 1M MgCl₂ 15 mM, 25 mM KCl, 4 mM DTT].

Aminoacylation of Total E. coli tRNA

Aminoacylation of total E. coli tRNA (1 mM) with all 20 amino acids (1 mM each) is performed by incubation for 10 minutes at 37° C. with ATP (5 mM) and the appropriate amount of S100 in 30 mM Tris-HCl pH 7.5, 15 mM 1M MgCl₂, 25 mM KCl, 4 mM DTT. To stop the reaction and recover the aminoacylated tRNAs one-tenth volume of 3M NaOAc pH 5.0 is added, and the reaction extracted two times with acid phenol, two times with chloroform, and precipitated with 2.5 volumes of ethanol. The total aminoacylated tRNAs are resuspended at a concentration of 1 mM in 0.2 M NaOAc pH 5.0.

Reductive Methylation of Total E. coli tRNA (Transformation)

Total aminoacylated tRNA 1 mM is mixed with o-nitrobenzaldehyde (10 mM) and sodium cyanoborohydride (10 mM) in 100 mM NaOAc pH 5.0 for 30 minutes. The N-monosubstituted tRNAs are recovered by ethanol precipitation. Repeating this procedure with formaldehyde instead of o-nitrobenzaldehyde provides the N-disubstituted tRNAs. Subsequent removal of the nitrophenyl group by UV irradiation for 10 minutes (at a distance of 10 cm) with a 550 watt, mercury vapor lamp (ACE glass) provides the desired mixture of N-Me-tRNAs.

### Example 2

### Use of Me-N-tRNAs in Generating Encoded Non-Standard Polymer Libraries

An in vitro translation mixture is combined with a complex pool of mRNA sequences, and an appropriate amount of bifunctional tRNA for making fusions Merryman, et al., Chem. & Biol. 9:741-746 (2002); Merryman and Bartel, U.S. Pat. No. 6,440,695). The translation mixture contains all of the factors required for in vitro translation (e.g., initiation factors, transformed tRNA analogues, and elongation factors) except for mRNA. Translation mixtures can be made by a number of standard methods. Translation is initiated by the addition of the complex pool of mRNA sequences. All of the members of the pool of mRNA sequences have a constant sequence at their 5′ end that permits them to be translated by the ribosome, an internal, randomized, polymer-coding segment and a UUU- and UUC-rich 3′ coding segment that recruits a bifunctional tRNA after translation of the randomized segment is completed. Each codon in the mRNA recruits a transformed tRNA analogue and the ribosome synthesizes a non-standard polymer from Me-N-amino acids. When the UUU- and UUC-rich 3′ coding segment (of each translating mRNA) the bifunctional-tRNA is used to fuse each polymer to its encoding mRNA. After fusion, disruption of the ribosomes with EDTA releases the encoded non-standard polymer library so that it can be purified and used.

### Example 3

### Transformation of Aminoacyl tRNAs for the In Vitro Selection of “Drug-Like” Molecules

Materials and Methods

Ribosomes, S150 enzyme fraction, aminoacyl-tRNAs, in vitro protein synthesis reactions and mRNAs were made and used as previously described (Merryman, C., et al., Chem. & Biol. 9:741-746 (2002)). Individual tRNA species were purchased from Subriden (Rolling Bay, Wash.) or Sigma (St. Louis, Mo.). Radiolabeled amino acids, amino acid mixtures, and formaldehyde were obtained from Moravek Biochemicals (Brea, Calif.), American Radiolabeled Chemicals (St. Louis, Mo.) or Sigma. Other materials were purchased from standard sources.

Transformation of Puromycin

N-methyl puromycin was synthesized by incubating 8 mM puromycin, 50 mM o-nitrobenzaldehyde, and 20 mM cyanoborohydride in buffer (100 mM NaOAc pH 5.0; 37° C.). After 30 min, 0.2 volumes of 100 mM formaldehyde was added to the reaction and the incubation continued (ambient; 30 min). The sparingly soluble nitrobenzaldehyde-puromycin derivatives were purified by centrifugation (16,000×g; 4° C.). Precipitates were suspended in ethanol, precipitated from water, and dissolved in ethanol containing 500 mM BME. Nitrobenzaldehyde adducts were removed by spotting the sample in the cap of a microfuge tube under a covered polystyrene Petri-dish, the dish placed on ice, and the sample exposed to UV for 7 min using a 550 watt Hanovia bulb with water jacket (Merryman, C., et al., Chem. & Biol. 9:741-746 (2002)). Assuming complete recovery, 6 nmol were banded on RP-18 F₂₅₄ TLC plates (Merck) and developed in 65% acetonitrile (pH 8.0).

Transformation of aminoacyl-tRNAs

N-methyl Glu, Ser and Val were synthesized while attached to their respective tRNAs by incubating 5 μM aminoacyl tRNA or 150 μM bulk tRNA with 30 mM o-nitrobenzaldehyde, and 20 mM cyanoborohydride in buffer A [50 mM KOAc, pH 5.0, 5 mM MgCl₂] at 37° C. for 30 min. After the initial incubation, 0.11 volumes of 100 mM formaldehyde was added and incubation continued (ambient; 30 min)—with radiolabeled formaldehyde (Sigma; ˜18 mM), the tRNAs were precipitated after the nitrobenzaldehyde reaction and used at 25 μM during the methylation step so that the final formaldehyde concentration could be maintained at 10 mM. The disubstituted tRNAs were purified by ethanol precipitation, suspended in buffer A with 50 mM BME, the nitrophenyl group removed as before, the tRNA precipitated, and dissolved in buffer A with 5 mM BME. Aminoacyl-tRNA ester bonds were cleaved in 250 mM KOH (30 min; 37° C.). Small aliquots (˜10 pmol) were spotted on Silica-gel-G TLC plates and developed in 80-90% ethanol containing 300 mM NH₄OAc (pH 5.0). For eTLC, the hydrolyzed N-methyl amino acids were mixed with authentic N-methyl amino acids (10 mM; Bachem), spotted on cellulose TLC plates (Merck) and electrophoresed (20 min; 6% formic acid, pH 1.5). For ninhydrin staining, equal volumes of 0.33% ninhydrin in tert-butanol and glacial acetic-water-pyridine (1:5:5) were mixed, applied to the plate, and the plate heated to 100° C. until developed.

Mock Treated Bulk tRNA

Naked bulk tRNA at 25 μM was treated according to the N-methylation protocol described above. Aminoacylation, and in vitro protein synthesis reactions using treated and untreated tRNA were performed as previously described (Merryman, C., et al., Chem. & Biol. 9:741-746 (2002)).

Acceptor Activity of Mock-Treated and N-methyl Aminoacyl tRNA

Dipeptides were formed by incubating various combinations of fMET-tRNA^{met} and tRNA with 1 μM ribosomes, 20 μg/ml pyruvate kinase, 3 μM each of IF1, IF2, and IF3, 4-20 μM EFTu, 10 mM phosphoenol pyruvate, 2 mM ATP, 500 μM each of GTP, CTP, and UTP and 2 μM mRNA in low Mg²⁺ buffer [10 mM Hepes, pH 7.5/6 mM MgOAc/150 mM NH₄Cl/0.6 mM spermine/0.4 mM spermidine/4 mM BME] or high Mg²⁺ buffer [10 mM Tris, pH 7.5/15 mM MgOAc/100 NH₄Cl/4 mM BME] for 10-30 min at 37° C. The mRNA 5′-GGGU UAA CUU UAG AAG GAG GUA AAA AAA AUG (NNN) UUU UUC UUU-3′ (SEQ ID NO: 2) was used to direct tRNA binding. The initiator methionine codon (AUG) is followed by an appropriate codon (NNN) for the respective amino acid (A=GCG, C=UGC, D=GAC, E=GAA, F=UUU, G=GGU, H=CAU, I=AUU, K=AAA, L=CUG, M=AUG, N=AAC, P=CCG, Q=CAG, R=CGG, S=UCU, T=ACC, V=GUC, W=UGG, Y=UAC). When [³⁵S]-Met labeled initiator tRNA was used, its concentration was 0.5-1 μM and the appropriate N-methyl aminoacyl tRNA was 1 μM (Val), 2 μM (Ser), 25 μM (bulk mock-treated tRNA) or 40 μM (bulk N-methylated tRNA). When unlabeled initiator tRNA was used its concentration was 1-1.5 μM and the appropriate N-methyl aminoacyl tRNA was 0.5 μM. After incubation, 0.3 volumes of 1 M KOH was added to cleave the dipeptides and unreacted amino acids from their respective tRNAs (30 min; 37° C.). The samples were spotted on cellulose TLC plates and electrophoresed (30-60 min; 20% acetic acid, 0.5% pyridine, pH 2.75).

Results

Synthesis of N-monomethyl Aminoacyl tRNA

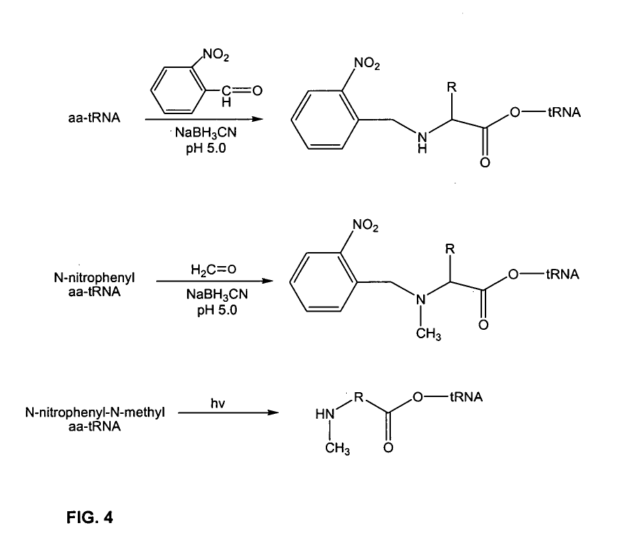

The steps used for N-methylation of enzymatically charged tRNA are outlined in FIG. 4. In the first step, the amino acids were protected from dimethylation by N-alkylation (Gareau, Y., et al, J. Org. Chem. 58:1582-1585 (1993)). The tRNA-bound amino acids were reacted o-nitrobenzaldehyde and sodium cyanoborohydride (FIG. 4A). Although the α-amine of an amino acid has the potential to undergo a second alkylation, with nitrobenzaldehyde this secondary substitution is sterically prevented (Ohfune, Y., et al, Chem. Letters 3:441-444 (1984)). Upon addition of the less hindered formaldehyde, a second reductive derivatization (this time methylation) proceeded smoothly (FIG. 4B). In a final step, the disubstituted intermediate (N-nitrophenyl-N-methyl aminoacyl tRNA) was exposed to UV light to remove the protective nitrophenyl group (FIG. 4C) and produce the desired tRNA with an N-monomethyl amino acid.

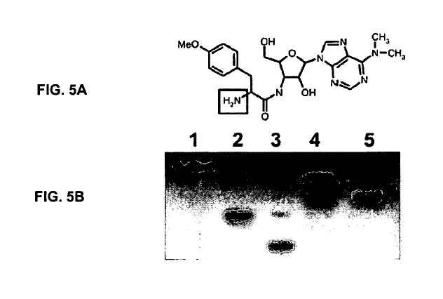

To optimize the chemistry and allow easily for product analysis, the above scheme was initially applied to puromycin, a minimal analog of aminoacyl tRNA that is chemically stable and commercially available on the gram scale (FIG. 5A). As shown by TLC (FIG. 5B), reductive alkylation with nitrobenzaldehyde decreased the mobility of puromycin (FIG. 5B, compare lanes 1 and 2) and subsequent addition of formaldehyde to the reaction resulted in conversion to the disubstituted intermediate (N-nitrophenyl-N-methyl puromycin; FIG. 5B, lane 3). N-monomethyl puromycin was generated by photoreversal of the disubstituted intermediate (FIG. 5B, lane 4) and confirmed by mass spectrometry (data not shown). If the protection step was not utilized, formaldehyde treatment led directly to dimethylation and this product had a distinct mobility (FIG. 5B, lane 5).

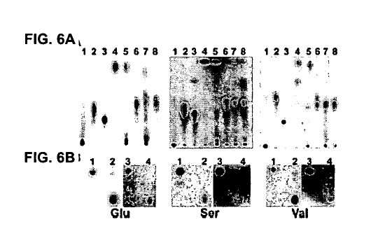

Next, to demonstrate the generality of the scheme we performed this chemical transformation on several different aminoacyl tRNAs (Glu, Ser, and Val). These diverse amino acids were selected to substantiate that the methylation protocol was fundamentally independent of the nature of the sidechain. As before, products from each of the individual steps were separated by TLC (FIG. 5A), but the ester linkage to the tRNA was hydrolyzed before running the TLC to allow direct observation of the amino acid moiety. As with puromycin, it was possible to separate the N-methyl products from the amino acids, disubstituted intermediates, and dimethyl amino acids. Reaction precursors and products were followed using either labeled amino acids or labeled formaldehyde allowing the analysis to establish that the reaction products contained an amino acid and a formaldehyde adduct as follows: lane 1, unhydrolyzed N-methyl [¹⁴C]-aminoacyl tRNA; lane 2, [¹⁴C]-aminoacyl tRNA; lane 3, N,N-dimethyl [¹⁴C]-aminoacyl tRNA; lane 4, N-nitrophenyl-N-methyl [¹⁴C]-aminoacyl tRNA; lane 5, N-nitrophenyl-N—[¹⁴C]-methyl aminoacyl tRNA; lane 6, N-methyl [¹⁴C]-aminoacyl tRNA; lane 7, N—[¹⁴C]-methyl aminoacyl tRNA; lane 8, N-methyl [¹⁴C]-aminoacyl tRNA from bulk tRNA. The equivalent (fast) migration of the disubstituted intermediates (N-nitrophenyl-N-methyl amino acids) when either labeled amino acid or labeled formaldehyde was used indicated that the desired double transformation had occurred (FIG. 6A, lanes 4 and 5). When only formaldehyde was used for derivatization, the dimethylated products were readily identified by their distinctive mobilities (FIG. 6A, lane 3). Finally, as expected for a nitrophenyl-protected amine, UV exposure shifted the mobilities of the disubstituted intermediates and the migrations of the photoreversed products were distinct from those of the amino acids, intermediates, or dimethylated controls (FIG. 6A, lanes 6 and 7). Again, irrespective of whether the amino acid or formaldehyde was labeled, the photoreversed products comigrated, consistent with the predicted formation of N-methyl amino acids. Without hydrolysis, the final products of the reaction remained at the origin demonstrating that the chemistry did not cleave the ester linkage between the tRNA and N-methyl amino acid (FIG. 6A, lane 1).

As a final control, methylation of the tRNA bound amino acids was verified by comparison to commercially available standards (FIG. 6B). Radiolabeled N-methyl Glu, Ser and Val were synthesized while bound to their respective tRNA, the aminoacyl-tRNA ester bond hydrolyzed, and the samples mixed with excess unlabeled N-methyl-amino acid standards. Here, the samples were analyzed by thin-layer electrophoresis (eTLC) to increase the resolution between the amino acids and their N-methyl analogs. These mixtures were then subjected to eTLC. Radiolabeled compounds (lanes 1 and 2) were visualized by phosphorimager analysis and the unlabeled standards (lanes 3 and 4) by ninhydrin staining: lane 1, amino acid; lane 2, N-methyl aminoacyl tRNA; lane 3, unlabeled amino acid standard; lane 4, unlabeled N-methyl amino acid standard.

Phosphorimager quantitation of the precursors and products indicated that greater than 95% of the radiolabeled amino acids were transformed into their N-methyl counterparts (FIG. 6B, lanes 1 and 2). Unlabeled standards were co-spotted, visualized with ninhydrin, photographed, and aligned by marks made on the thin-layer plates with radiolabeled dyes (FIG. 6B, lanes 3 and 4). The N-monomethyl amino acids were faintly stained by a modified ninhydrin protocol that allowed for establishment of their mobility (see Materials and Methods).

Transformation of Bulk tRNA

An important aspect of the methods described herein is their potential to simultaneously alter many amino acids after an enzymatic aminoacylation step is used to attach them to their respective tRNAs. By following this methodology, the need to purify individual tRNA species and subsequently subject each tRNA to a multistep misacylation protocol can be avoided. As shown in FIG. 6A, lane 8, transformation produced N-methyl amino acids from bulk tRNA as readily as from individually purified tRNAs. For these reactions, bulk E. coli tRNA was charged with unlabeled amino acids and the single radiolabeled amino acid of interest. With each of the amino acids directly tested, N-methyl products were efficiently produced and it seems probable that the remaining 18 N-methyl aminoacyl tRNAs were synthesized concurrently (proline should return to its original form after deprotection).

Activity of N-monomethyl Aminoacyl tRNA

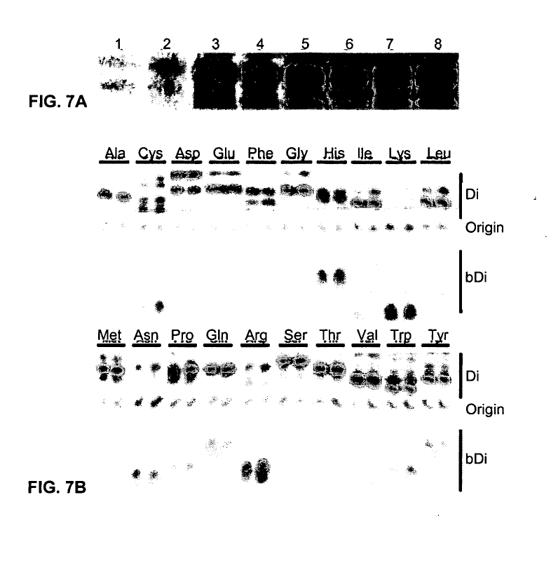

A key requirement for the synthesis of libraries of non-standard polymers is that the transformed tRNAs be substrates for the ribosome. This first will depend on the methylation chemistry itself not inactivating the tRNA body for interaction with the protein synthesis machinery. Although the standard nucleotides are not expected to undergo reductive alkylation, the potential for RNA folding to introduce uniquely reactive environments and the presence of primary and secondary amines in the post-transcriptionally modified nucleotides of tRNA suggested that unwanted modifications were likely. Initial experiments with radiolabeled formaldehyde and uncharged tRNA indicated that reductive methylation resulted in about one undesired modification per tRNA. Mock reactions with deacylated tRNA were performed to examine whether such side reactions affected the ability of tRNA to function properly in translation. As shown in FIG. 7A, translation of an 88-amino acid long fragment of β-galactosidase proceeded at the same rate and to the same extent with both treated and untreated tRNA. As substrates for translation, ribosomes were supplied untreated bulk aminoacyl tRNA (lanes 2, 3, 5, and 7) or naked bulk tRNA that had been aminoacylated and treated according to the N-methylation protocol (lands 4, 6, and 8). Aminoacylation of the tRNAs was performed in the translation mix. Translation products labeled with [³⁵S]-met were resolved on a 12% nuPAGE gel (Invitrogen) and visualized by phosphorimager analysis. Lane 1, contained no exogenous tRNA and was incubated for 27 min; lane 2, contained no mRNA and was incubated for 27 min; lanes 3 and 4, translation products after 3 min; lanes 5 and 6, products after 9 min; lanes 7 and 8, products after 27 min. Furthermore, for each amino acid, mock-treated tRNA was just as competent as untreated tRNA in forming fMet-labeled dipeptides on the ribosome (FIG. 7B). Thus, although formaldehyde is incorporated into the body of tRNA (i.e., observe the radiolabeled formaldehyde which remains at the origin with tRNA; FIG. 6A, lane 5 and 7) this modification had no detectable consequences for tRNA function. This is presumably because the modifications are distributed randomly and/or occur at positions that are not critical for overall tRNA function.

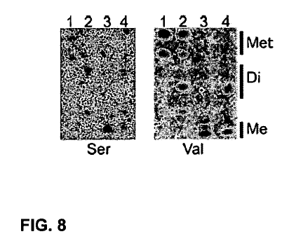

Whether monomethylated Ser and Val were effectively incorporated by the ribosome was then investigated. This was examined by testing the ability of the transformed amino acids to form dipeptides encoded by the supplied mRNA. Initiation complexes containing fMet-tRNA and the appropriate mRNA were formed, and each of the N-methyl aminoacyl tRNAs was added. Production of fMet-N-methyl dipeptides indicated that the transformed tRNAs were recruited by the ribosome and were active in peptide bond formation (FIG. 8). This documentation of activity is important for two reasons. First, as above, it confirms that chemical transformation does not meaningfully alter the tRNA itself. Second, the production of dipeptides that contain labeled N-methyl amino acids (FIG. 8, lane 4) provides clear evidence that such substrates can be polymerized by the ribosome.

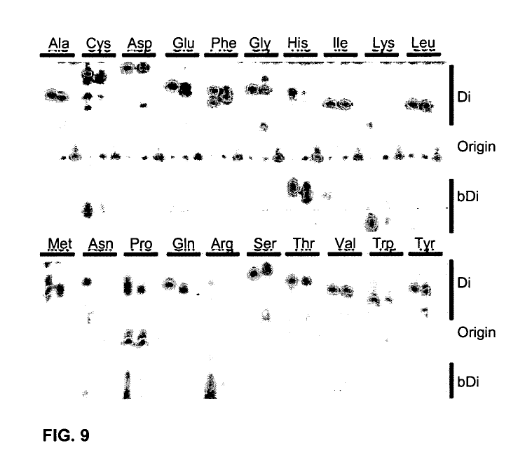

To examine the acceptor activity of each of the N-methyl amino acids, [³⁵S]-Met labeled dipeptides were produced from initiation complexes that were supplied bulk tRNA and an appropriate mRNA. The amount of dipeptide formed when normal tRNA, N-methyl tRNA or dimethylated tRNA was used as an acceptor substrate is shown in FIG. 9. In general, when compared to the amount of dipeptide formed with normal tRNA, the extent of reaction with N-methylated amino acids was lower but quite respectable (FIG. 9, compare the first and second lanes for each amino acid). Several amino acids were efficiently incorporated (>50%; N-methyl-Ala, Phe, His, Ile, Leu and Val) whereas a number of others were incorporated less well but with an activity of at least 30% in comparison to the normal amino acid (N-methyl-Cys, Asp, Gly, Met, Gln, Ser and Thr). While N-methyl-Glu, Lys, Asn, Arg and Trp were effectively produced by the method described herein, they were less effective substrates (<15%) in the dipeptide assay. While not wishing to be bound by theory, N-methyl-glu, lys, asn, arg, and trp appeared to be taken up by the ribosome less well than other N-methyl amino acids. However, it is believed that N-methyl- glu lys, asn, arg and trp tRNAs that are selected by the ribosome can function in ribosome-directed translation.

Because product analysis for each of the dipeptide products was not feasible, we used a number of criteria to establish that the primary product species was authentic. First, in a number of cases, the migration of the dipeptide formed with N-methylated tRNA differed from the migration of the dipeptide product formed with untreated aminoacyl tRNA which suggested that a methyl group was present and that it altered the mobility of the dipeptide (FIG. 9; compare the first and second lanes, for Ala, His, Gln, Ser, Tyr). Next, it was noted that N-methyl aminoacyl tRNAs were almost completely inactive in dipeptide formation at conditions where normal tRNA was fully functional-normal and mock treated tRNAs were efficient substrates at both low (6 mM) and high (15 mM) magnesium concentration while the methylated aminoacyl tRNAs were incorporated only at 15 mM magnesium. Because the transformation procedure has no effect on acceptor activity when it is performed before bulk tRNA is aminoacylated (FIG. 9), the magnesium dependence and reduced acceptor activity of the tRNAs when transformation follows aminoacylation must result from derivatization of the amino acids themselves. Finally, it was established that alkylation was efficient with each of the amino acids by leaving o-nitrobenzaldehyde out of the transformation reaction (so that the tRNA products carried inactive N,N-dimethyl amino acids) and showing that no dipeptide was produced from any of the mRNA templates (FIG. 9, third lane of each amino acid). Thus, it appears that all of the amino acids are efficiently alkylated.

Discussion

Demonstrated herein is a procedure generated N-monomethylated tRNAs from their aminoacylated tRNA precursors under mildly acidic, aqueous conditions. Most of the N-methylated aminoacyl tRNA substrates had significant acceptor activity on the ribosome in peptidyl transferase assays. As discussed above, this approach is particularly well suited for synthesizing complex libraries of non-standard polymers comprising a methylated backbone due to the ease with which the non-standard aminoacyl tRNAs are generated. In conjunction with ribosome based encoding schemes, this approach opens the prospect that methylated peptides with desirable functions can be identified by in vitro selection from diverse populations (˜10¹³).

The ribosome-based production of methylated peptides depends on producing non-standard aminoacyl tRNAs that reliably incorporate specific N-methyl amino acids in response to specific codons. The methods described herein use tRNAs that are charged by highly accurate synthetases, and thus the expected code (N-methyl Ser at Ser codons, etc) has the potential to be essentially error free. There are, however, mechanisms by which a chemical transformation could disrupt the ability of a tRNA to incorporate the correct monomer into a growing chain. For example, incomplete methylation of an amino acid or its sidechain would result in a degenerate tRNA population bearing normal and transformed amino acids. Thus, each mRNA sequence would potentially code for a wide variety of mixed polymers rather than a single methylated peptide. This potential problem is exacerbated with a poorly used N-methyl amino acid because the unmethylated version, even if present at low concentrations, would be preferentially incorporated by the ribosome. However, the extent of methylation observed (>95%) with three rather different aminoacyl tRNAs suggests that N-methylation is quite robust, and it is expected that a second round of formaldehyde treatment can increase the yields to better than 99.5%. Furthermore, as no dipeptides are formed from any of the dimethylated tRNAs (Fig. third lane for each amino acid), complete alkylation appears to be independent of the amino acid, and therefore, few problems are expected from untransformed amino acids in the population.

The efficiency of the peptidyl transferase reaction and the apparent homogeneity of the dipeptide product varied across the range of amino acids. In the simplest cases, dipeptide products formed from N-methyl-Ala, His, Gln, Ser and Tyr had slightly different mobilities than the dipeptides formed from the corresponding unmethylated amino acids. These changes in migration are anticipated based on altered chemical properties resulting from the acquisition of a non-polar methyl group. The fact that a single product was observed for these amino acids argues strongly that the product was the N-methylated version of the desired dipeptide. For a second group of amino acids (Asp, Gly, Ile, Leu, Thr and Val) the unmethylated and methylated dipeptide products migrated similarly. When assayed directly, radiolabeled N-methyl Val was incorporated into dipeptide (FIG. 8.) and was fully methylated in bulk tRNA (FIG. 6, lane 8). It seems probable that the other dipeptides also contain N-methyl amino acids but, like N-methyl Val, their incorporation does not alter the mobility of their respective product. This is supported by the fact that following the methylation protocol, all of the N-methyl amino acids were efficiently incorporated only when additional Mg²⁺ was supplied to induce productive interaction with the ribosome. This condition was unnecessary with normal or mock treated tRNA and thus, the effects must result from alteration of the amino acids themselves. Suitable conditions for ribosome-directed translation using the aminoacyl tRNA analogues of the present invention is described in Merryman and Green, Chem & Biol. Online publication Mar. 18, 2004 DOI: 10.1016/S1074552104000857 on the world wide web at chembiol.com/content/future). While not wishing to be bound by theory, increasing the concentration of Mg²⁺ allows the ribosome to use unusual tRNAs such as the aminoacyl tRNA analogues of the present invention. Increasing the concentration of Mg²⁺ can also increase the error rate of the ribosome. One of ordinary skill in the art would be able to select a suitable concentration of Mg²⁺ to broaden the specificity of the ribosome while maintaining a low or acceptable error rate.

Multiple dipeptide products were formed from a third group of modified amino acids (Cys, Phe, and Met) indicating that there was some miscoding for these codon:anticodon pairs. In these cases, while the correct N-methylated aminoacyl tRNA appeared to be used, inappropriate, presumably near-cognate, tRNAs were selected by the ribosome at some rate. Such miscoding was not wholly unexpected as the elevated magnesium concentrations that were required to recruit N-methyl aminoacyl tRNAs onto the ribosome are known to decrease fidelity (Thompson, R. C., et al. J. Biol. Chem. 256:6676-6681 (1980)). It remains possible that codons not tested here might exhibit lower levels of miscoding. These fidelity issues might also be addressed experimentally by further optimization of the reaction conditions. Indeed, when more tRNA was added to these reactions, the overall level of miscoding decreased in agreement with previous results (Wagner, E. G., et al., Mol. Gen. Genet. 185:269-274 (1982)). Fortunately, unlike recent methods that propose the use of chemically charged tRNA (Merryman, C., et al., Chem. & Biol. 9:741-746 (2002); Forester, A. C., et al., Proc. Natl. Acad. Sci. USA 100:6353-6357 (2003); and Frankel, A., et al., Chem. & Biol. 10:1043-1050 (2003)), the methods described herein can easily supply excess tRNA to limit substrate depletion and the associated problems of miscoding and translation inhibition via the buildup of uncharged tRNA.

N-methyl-Glu, Lys, Asn, Arg and Trp were not as efficiently incorporated. The inefficiently incorporated N-methyl amino acids can be left underivatized in a tRNA substrate mix. While attention was focused on evaluating the potential to generate universally methylated peptides, the described methods provide the basic tools necessary to generate libraries from combinations of normal and N-methyl amino acids. Selective methylation can be envisioned by charging bulk tRNA with a subset of amino acids and after transformation of these residues, the remaining naked tRNAs would be charged with normal amino acids. Because N-methyl amino acids do not appear to be removed by the editing function of crude aminoacyl tRNA synthetases, the second round of aminoacylation will likely not require purified enzymes.

Beyond providing access to libraries of methylated peptides, the methods described herein can be extended to encompass other backbones. This can be accomplished by using different aldehydes or ketones during the reductive alkylation step to add other moieties to the α-amine of the aminoacyl tRNAs.

Modification of specific amino acid sidechains can easily extend the properties of the libraries described herein by expanding the range of available monomers. For example, derivatizable sidechains such as lysine or cysteine could be used to mount diverse groups on normal or suppressor tRNAs for inclusion within libraries of methylated peptides. In previous evolutionary experiments, similar manipulations have produced peptide-drug conjugates whose activity is 100-fold better than the parent compound (Li, S. and Roberts, R. W. Chem. Biol. 10: 233-239 (2003)). With methylated amino acids, the pendant moieties are mounted on a backbone that has the potential to enhance the bioavailability and biostability of the parent compound in addition to improving its affinity or target selectivity. It therefore might prove shrewd to devise selective schemes that are aimed at incorporating properties such as improved bioavailability and/or improved biostability that are lacking in existing or abandoned compounds.

### Example 4

### Protease Resistance of Random N-Methylated Non-standard Polymers

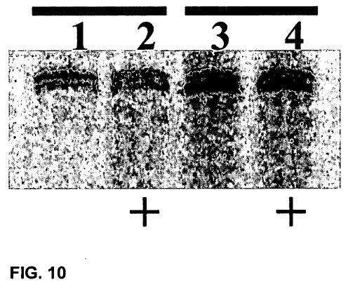

In vitro translation reactions were used to synthesize non-standard polymers from total E. coli tRNA that had been charged with the normal 20 amino acids and then transformed as described above, to produce bulk tRNA analogues comprising a tRNA and N-methylated amino acid (except for tRNA^{pro} which returns to its original form). The N-methyl-aminoacyl tRNAs were used to translate a mRNA pool containing five completely randomized codons, resulting in the production of randomized pentapeptides with methylated backbones. The mRNA (ggguuaacuuuagaaggagguaaaaaaaAUG nnn nnn nnn nnn nnn uuuuucuuu, SEQ ID NO: 3) was used to direct translation. The initiator methionine codon (AUG) is in capital letters, and the five randomized codons are represented by the five “nnn” triplets, which comprise the complete set of 64 possible codons. Translation products were labeled at their amino termini by initiating translation with a [³⁵S]-fMet containing initiator tRNA, and translation products were covalently fused to tRNA after they were completed (See Merryman et al, A Bifunctional tRNA for in vitro selection, Chem Biol. 9:741-6 (2002)). Proteinase K (0.02 mg/ml) was used to hydrolyze the tRNA-bound peptide products [10 min; 37° C.; 50 mM Tris, pH 7.5; 150 mM NaCl]. Untreated (FIG. 10, lanes 1, and 3) and protease treated samples (FIG. 10, lanes 2, and 4) were separated by polyacrylamide gel electrophoresis and visualized by phosphorimager analysis. Control reactions contained translation products that were generating using total tRNA that had been charged but not methylated (FIG. 10, lanes 1 and 2; normalized by diluting 4 fold). Comparison of lanes 2 (randomized peptides) and 4 (randomized N-methylated peptides) demonstrates that the non-standard polymers are protease resistant.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of producing an aminoacylated tRNA analogue represented by the following structural formula: 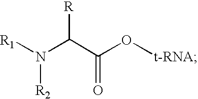
wherein:
R is an amino acid side chain;
R₁ is H or an amine protecting group;
R₂ is a group other than H such that the compound represented by the following formula acts as an acceptor substrate and a donor substrate for ribosome-directed translation: 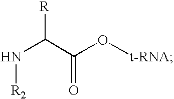
said method comprising the step of reacting a starting compound with a reagent capable of converting R₁—NH—R″ to R₁—NR₂—R″, wherein R″ is an organic moiety, under conditions suitable for said reaction, wherein:
the starting compound is represented by the following structural formula:

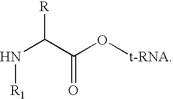

2. The method of claim 1 wherein the protecting group is a nitrophenyl group.

3. The method of claim 1 wherein R₂ is a methyl group.

4. The method of claim 3 wherein the amino acid side chain is from an amino acid selected from the group consisting of: alanine, arginine, asparagine, aspartic acid, cysteine, glutamine, glutamic acid, glycine, histidine, isoleucine, lysine, methionine, phenylalanine, serine, threonine, tryptophan, tyrosine, valine, and analogues thereof.

5. A method of preparing the aminoacylated tRNA analogue of claim 1 represented by the following structural formula: 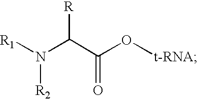
wherein:
R is an amino acid side chain;
R₁ is H or an amine protecting group;
R₂ is an alkyl group such that the compound represented by the following formula acts as an acceptor substrate and a donor substrate for ribosome-directed translation; 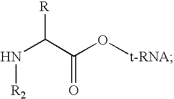
said method comprising the step of alkylating a starting compound represented by the following structural formula:

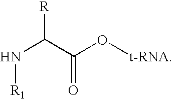

6. The method of claim 5 wherein the starting compound is alkylated with an alkyating agent.

7. The method of claim 6 wherein the alkylating agent is represented by R₂X, wherein X is a leaving group.

8. The method of claim 5 wherein the starting compound is alkylated by reductively aminating R₂CHO with the starting compound.

9. The method of claim 8 wherein the starting compound is alkylated by reacting the starting compound with R₂CHO and an imine reducing agent.

10. The method of claim 6 wherein R₂ is CH₃ and the imine reducing agent is sodium cyanoborohydride.

11. The method of claim 1, additionally comprising the step of deprotecting the product compound to form an aminoacylated tRNA analogue represented by the following structural formula:

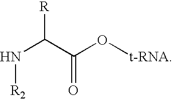

12. A method for producing an aminoacyl tRNA analogue comprising a tRNA and a modified amino acid, comprising substituting an aminoacyl tRNA under conditions in which an α-amino group of the aminoacyl tRNA is monosubstituted with a group other than H, such that the aminoacyl tRNA analogue acts as an acceptor substrate and a donor substrate for ribosome-directed translation, thereby producing the aminoacyl tRNA analogue.

13. The method of claim 12, wherein the conditions under which the α-amino group of the aminoacylated tRNA is monosubstituted comprise protecting the α-amino group from disubstitution, thereby producing a protected aminoacyl tRNA analogue.

14. The method of claim 13, wherein the protected aminoacyl tRNA is produced by contacting the aminoacyl tRNA with o-nitrobenzaldehyde and sodium cyanoborohydride, thereby N-alkylating the amino acid of the aminoacyl tRNA.

15. The method of claim 14, further comprising deprotecting the protected aminoacyl tRNA.

16. The method of claim 15 wherein the protected aminoacyl is deprotected by photoreversal.

17. The method of claim 12, wherein alkylation is used to substitute the α-amino group of the protected aminoacylated tRNA.

18. The method of claim 17, wherein the alkylation of the α-amino group produces an aminoacylated tRNA analogue comprising an N-methylated amino acid.

19. A method for producing an aminoacyl tRNA analogue comprising a tRNA and a modified amino acid which acts as an acceptor substrate and a donor substrate for ribosome-directed translation, comprising:
(a) combining a tRNA, an amino acid, and an aminoacyl tRNA synthetase under conditions in which an aminoacylated tRNA is formed; and
(b) substituting the aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation;
thereby producing the aminoacyl tRNA analogue.

20. A method for producing a population of tRNAs comprising aminoacyl tRNAs and aminoacyl tRNA analogues, wherein the aminoacyl tRNA analogues comprise a tRNA and a modified amino acid such that the aminoacyl tRNA analogues act as acceptor substrates and donor substrates for ribosome-directed translation, comprising:
(a) combining a plurality of tRNAs that are specific for a plurality of amino acids, amino acids which are specific for a subset of the tRNAs, and aminoacyl tRNA synthetases, under conditions in which the subset of tRNAs are aminoacylated, thereby forming a mixture of tRNAs that are aminoacylated and tRNAs that are not aminoacylated;
(b) substituting the tRNAs that are aminoacylated of (a) under conditions in which an α-amino group of each aminoacylated tRNA is monosubstituted with a group other than H, thereby forming aminoacyl tRNA analogues which act as acceptor substrates and donor substrates for ribosome-directed translation; and
(c) contacting the mixture of (b) with amino acids that are specific for the tRNAs that are not aminoacylated, under conditions in which aminoacylated tRNAs are formed;
thereby producing the population of tRNAs comprising aminoacyl tRNAs and aminoacyl tRNA analogues.

21. A method for producing a non-standard polymer comprising:
(a) producing an aminoacyl tRNA analogue comprising substituting an aminoacyl tRNA under conditions in which an α-amino group of the aminoacyl tRNA is monosubstituted with a group other than H, such that the aminoacyl tRNA analogue acts as an acceptor substrate and a donor substrate for ribosome-directed translation;
(b) combining:
(i) an encoding nucleic acid;
(ii) the aminoacyl tRNA analogue of (a); and
(iii) an in vitro translation mixture;
(c) maintaining the combination of (b) under conditions in which the aminoacyl tRNA analogue is used in the translation of the encoding nucleic acid,
thereby producing a non-standard polymer.

22. The method of claim 21, further comprising forming a stable complex that includes the non-standard polymer and its encoding nucleic acid, such that the non-standard polymer and its encoding nucleic acid can be co-isolated.

23. The method of claim 22, wherein the stable complex further comprises a tRNA having a Y base, wherein the Y base has been activated to covalently crosslink said tRNA to the encoding nucleic acid.

24. The method of claim 21, wherein the conditions under which the α-amino group of the aminoacylated tRNA is monosubstituted comprise protecting the α-amino group from disubstitution, thereby producing a protected aminoacyl tRNA analogue.

25. The method of claim 22, wherein the protected aminoacyl tRNA is produced by contacting the aminoacyl tRNA with o-nitrobenzaldehyde and sodium cyanoborohydride, thereby N-alkylating the amino acid of the aminoacyl tRNA.

26. The method of claim 25, further comprising deprotecting the protected aminoacyl tRNA.

27. The method of claim 26 wherein the protected aminoacyl is deprotected by photoreversal.

28. The method of claim 24, wherein alkylation is used to substitute the α-amino group of the protected aminoacylated tRNA.

29. The method of claim 28, wherein the alkylation of the α-amino group produces an aminoacylated tRNA analogue comprising an N-methylated amino acid.

30. The method of claim 21, wherein alkylation is used to monosubstitute the α-amino group of the aminoacylated tRNA.

31. The method of claim 21, wherein the in vitro translation mixture of (b)(iii) includes aminoacyl tRNA other than the aminoacyl tRNA of step (a).

32. A method for producing a library comprising non-standard polymers comprising:
(a) producing an aminoacyl tRNA analogue comprising substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue;
(b) combining:
(i) a population of encoding nucleic acids;
(ii) the aminoacyl tRNA analogue of (a); and
(iii) an in vitro translation mixture; and
(c) maintaining the combination of (b) under conditions in which the aminoacyl tRNA analogue is used in the translation of the encoding nucleic acids,
thereby producing a library comprising non-standard polymers.

33. The method of claim 32, wherein the combination of (b) is maintained under conditions suitable to form stable complexes that include the non-standard polymers and their encoding nucleic acids, such that a non-standard polymer having a desired property and its encoding nucleic acid can be co-isolated.

34. The method of claim 33, wherein the combination of (b) further comprises a tRNA having a Y base, wherein the Y base has been activated to covalently crosslink said tRNA to the encoding nucleic acid.

35. The method of claim 32, wherein the conditions under which the α-amino group of the aminoacylated tRNA is monosubstituted comprise protecting the α-amino group from disubstitution, thereby producing a protected aminoacyl tRNA analogue.

36. The method of claim 35, wherein the protected aminoacyl tRNA is produced by contacting the aminoacyl tRNA with o-nitrobenzaldehyde and sodium cyanoborohydride, thereby N-alkylating the amino acid of the aminoacyl tRNA.

37. The method of claim 36, further comprising deprotecting the protected aminoacyl tRNA.

38. The method of claim 37 wherein the protected aminoacyl is deprotected by photoreversal.

39. The method of claim 32, wherein alkylation is used to substitute the α-amino group of the protected aminoacylated tRNA.

40. The method of claim 39, wherein the alkylation of the α-amino group produces an aminoacylated tRNA analogue comprising an N-methylated amino acid.

41. The method of claim 33, wherein the in vitro translation mixture of (b)(iii) includes aminoacyl tRNA other than the aminoacyl tRNA of step (a).

42. A method for identifying a non-standard polymer having a desired activity, comprising:
(A) producing a library comprising non-standard polymers comprising:
(i) producing an aminoacyl tRNA analogue comprising substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue;
(ii) combining:
(a) a population of encoding nucleic acids;
(b) the aminoacyl tRNA analogue of (A)(i); and
(c) an in vitro translation mixture; and
(iii) maintaining the combination of (A)(ii) under conditions in which the aminoacyl tRNA analogue is used in the translation of the encoding nucleic acids, thereby producing a library comprising non-standard polymers; and
(B) selecting a member of the library that exhibits a desired activity, thereby identifying a non-standard polymer having a desired activity.

43. The method of claim 42, wherein the combination of (A)(ii) is maintained under conditions suitable to form stable complexes that include the non-standard polymers and their encoding nucleic acids, such that a non-standard polymer having a desired property and its encoding nucleic acid can be co-isolated.

44. The method of claim 42, further comprising:
(C) amplifying the encoding nucleic acid of the selected library member; and
(D) repeating steps (A), (B), and optionally (C), using the amplified nucleic acid as the population of encoding nucleic acids in step (A)(ii).

45. The method of claim 44, wherein mixture of (A)(ii) further comprises a tRNA having a Y base, wherein the Y base has been activated to covalently crosslink said tRNA to the encoding nucleic acid.

46. The method of claim 42, wherein the conditions under which the α-amino group of the aminoacylated tRNA is monosubstituted comprise protecting the α-amino group from disubstitution, thereby producing a protected aminoacyl tRNA analogue.

47. The method of claim 46, wherein the protected aminoacyl tRNA is produced by contacting the aminoacyl tRNA with o-nitrobenzaldehyde and sodium cyanoborohydride, thereby N-alkylating the amino acid of the aminoacyl tRNA.

48. The method of claim 47, further comprising deprotecting the protected aminoacyl tRNA.

49. The method of claim 48 wherein the protected aminoacyl is deprotected by photoreversal.

50. The method of claim 46, wherein alkylation is used to substitute the α-amino group of the protected aminoacylated tRNA.

51. The method of claim 50, wherein the alkylation of the α-amino group produces an aminoacylated tRNA analogue comprising an N-methylated amino acid.

52. The method of claim 42, wherein the in vitro translation mixture of (A)(ii)(c) includes aminoacyl tRNA other than the aminoacyl tRNA of step (A)(i).

53. An aminoacyl tRNA analogue comprising a tRNA and a modified amino acid, wherein an α-amino group of the amino acid is monosubstituted with a lower alkyl group, and wherein if the α-amino group is monosubstituted with a methyl group, the amino acid is other than leucine, glycine, phenylalanine or alanine, wherein the aminoacyl tRNA analogue acts as an acceptor substrate and a donor substrate for ribosome-directed translation.

54. The aminoacyl tRNA analogue of claim 53, wherein the lower alkyl group is a methyl group.

55. The aminoacyl tRNA analogue of claim 53, wherein the amino acid is selected from the group consisting of: alanine, arginine, asparagine, aspartic acid, cysteine, glutamine, glutamic acid, glycine, histidine, isoleucine, lysine, methionine, phenylalanine, serine, threonine, tryptophan, tyrosine, valine, and analogues thereof.

56. A population of aminoacyl tRNA analogues wherein each aminoacyl tRNA analogue comprises a tRNA and a modified amino acid wherein an α-amino group of each amino acid is monosubstituted with a lower alkyl group, and wherein the amino acids include alanine, arginine, asparagine, aspartic acid, cysteine, glutamine, glutamic acid, glycine, histidine, isoleucine, lysine, methionine, phenylalanine, serine, threonine, tryptophan, tyrosine, and valine, wherein the aminoacyl tRNA analogues act as acceptor substrates and donor substrates for ribosome-directed translation.

57. An aminoacyl tRNA analogue comprising a tRNA and a modified amino acid, wherein an α-amino group of the amino acid is monosubstituted with a protecting group selected from the groups consisting of: o-nitrophenyl and substituted versions thereof.

58. The aminoacyl tRNA analogue of claim 57, wherein the amino acid is selected from the group consisting of: alanine, arginine, asparagine, aspartic acid, cysteine, glutamine, glutamic acid, glycine, histidine, isoleucine, lysine, methionine, phenylalanine, serine, threonine, tryptophan, tyrosine, valine, and analogues thereof.

59. An aminoacyl tRNA analogue comprising a tRNA and a modified amino acid, wherein an α-amino group of the amino acid is substituted with a protecting group and a lower alkyl group.

60. The aminoacyl tRNA analogue of claim 59, wherein the lower alkyl group is a methyl group.

61. The aminoacyl tRNA analogue of claim 60, wherein the amino acid is selected from the group consisting of: alanine, arginine, asparagine, aspartic acid, cysteine, glutamine, glutamic acid, glycine, histidine, isoleucine, lysine, methionine, phenylalanine, serine, threonine, tryptophan, tyrosine, valine, and analogues thereof.

62. The aminoacyl tRNA analogue of claim 59, wherein the protecting group is selected from the groups consisting of: o-nitrophenyl and substituted versions thereof.

63. A method for producing a non-standard polymer comprising:
(A) combining:
(i) an encoding nucleic acid;
(ii) an aminoacyl tRNA analogue produced by substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue; and
(iii) an in vitro translation mixture; and
(B) maintaining the combination of (A) under conditions in which the aminoacyl tRNA analogue is used in the translation of the encoding nucleic acid,
thereby producing a non-standard polymer.

64. A method for producing a library comprising non-standard polymers comprising:
(A) combining:
(i) a population of encoding nucleic acids;
(ii) an aminoacyl tRNA analogue produced substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue; and
(iii) an in vitro translation mixture; and
(B) maintaining the combination of (A) under conditions in which the aminoacyl tRNA analogue is used in the translation of the encoding nucleic acids,
thereby producing a library comprising non-standard polymers.

65. A method for identifying a non-standard polymer having a desired activity, comprising:
(A) producing a library comprising non-standard peptides comprising:
(I) combining:
(a) a population of encoding nucleic acids;
(b) an aminoacyl tRNA analogue produced by substituting an aminoacyl tRNA under conditions in which an α-amino group of the aminoacylated tRNA is monosubstituted with a group other than H, such that the aminoacyl tRNA analogue acts as an acceptor substrate and a donor substrate for ribosome-directed translation, thereby producing the aminoacyl tRNA analogue; and
(c) an in vitro translation mixture;
(II) maintaining the combination of (A)(D under conditions in which the aminoacyl tRNA analogue is used in the translation of the encoding nucleic acids,
thereby producing a library comprising non-standard polymers;
(B) selecting a member of the library that exhibits a desired activity, thereby identifying a non-standard polymer having a desired activity.


SEQUENCE LISTING (3 sequences)

SEQ ID NO: 1 - RNA, 76 nt, unknown
  chemically synthesized
gcggauuuagcucagddgggagagcgccagacugaayancuggagguccugugnncgauccacagaauucgcacca

SEQ ID NO: 2 - RNA, 43 nt, unknown
  chemically synthesized mRNA used to direct translation
ggguuaacuuuagaaggagguaaaaaaaaugnnnuuuuucuuu

SEQ ID NO: 3 - RNA, 55 nt, unknown
  chemically synthesized mRNA used to direct tRNA binding
ggguuaacuuuagaaggagguaaaaaaaaugnnnnnnnnnnnnnnnuuuuucuuu